SEQUENCE LISTING (16 sequences)

SEQ ID NO: 1 - DNA, 2465 bp, Homo sapiens
gttgaagctcggcgctcgggttacccctgcagcgacgccccctggtcccacagataccactgctgctcccgccctttcgctcctcggccgcgcaatgggcacccgcgacgacgagtacgactacctctttaaagttgtccttattggagattctggtgttggaaagagtaatctcctgtctcgatttactcgaaatgagtttaatctggaaagcaagagcaccattggagtagagtttgcaacaagaagcatccaggttgatggaaaaacaataaaggcacagatatgggacacagcagggcaagagcgatatcgagctataacatcagcatattatcgtggagctgtaggtgccttattggtttatgacattgctaaacatctcacatatgaaaatgtagagcgatggctgaaagaactgagagatcatgctgatagtaacattgttatcatgcttgtgggcaataagagtgatctacgtcatctcagggcagttcctacagatgaagcaagagcttttgcagaaaagaatggtttgtcattcattgaaacttcggccctagactctacaaatgtagaagctgcttttcagacaattttaacagagatttaccgcattgtttctcagaagcaaatgtcagacagacgcgaaaatgacatgtctccaagcaacaatgtggttcctattcatgttccaccaaccactgaaaacaagccaaaggtgcagtgctgtcagaacatctaaggcatttctcttctcccctagaaggctgtgtatagtccatttcccaggtctgagatttaaatatatttgtaattcttgtgtcacttttgtgttttattacttcatacttatgaatttttccatgtcctaagtcttttgattttagctttataaaatcatccacttgtcccgaatgactgcagctttttttcatgctatggcttcactagccttagtttaataaactgaatgtttggattcctcagttattgtttacttttcatcatggaagcctgtcactgtatgtaggacataatagaacttgatcacttgaagctcagacctattggtcttgatcaaatcaaactaagaagaccttagaaataagctaccattttgccacagagcagcttataggtaatacactcttctctcagtgcagtgtacatttccacaaatctaagaattgccctataaacatagcaggattttgagagcttgaaaattttccattattctggacatgaatttctaaaatgccttaataggtttatgtagttgagtaaattttgttttttaatttttgtaagcatcaaagttgattagagaggggggcactttttctggagaattctcttagtaaacacaaaagattgttacggtttcattagtagtatggttgtggggccataagttaaacagtgctgcctggtaggctgggaactgaagagacttgtggtattccatctcgggtgcctctgttggcaatgatcaggcagcccaaaagatttaaatgatctataataatttccaagcggtagattatgtggcattttattgctcaggcaataattggtttaatgctggtagtgtcaaattttgtcttagaaccttccagtaagtgaaatacaacctagttttatcaccatatccaccagcaggcatggataattattttaacaatgctaatatttgagttttgcagtatattatagaatatagtccagttaaatctttggtttcagtatgtctgaagagtacagtgagaggttaatttctgctcaagtggtaccacttaaaggcatgtattcttttagtatgtaaaatgaaatagtaccttgagtttaaatagaatgcatttaggcattgtagagatctgaaatagttttcttccactacattgttgaaatcaatgaagcaattagtttctcattcagaaatgtgcacactaatatttagttttgctttctcgtggataatattaagcacttactctgcagtttcctggaagttgtgtcaactgcagtgatactattcaggatggtgggaaatccccaaaaatatgtatgtgtgggcttgcttagattactatatttcatagttaatcttttgtctcttgcggtgctcatgatgtgtggggcacacggaaggcattgctgtagtcagtcattttggttttcttctatagccattttattattttagtgtattagttatgaagataatattatctatttgtaaattgctactttgtattttatgcatgctctgtaatttgatttttttttagttattgatttggattatattcacattctaataaacagttatagggggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 216 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNGLSFIETSALDSTNVEAAFQTILTEIYRIVSQKQMSDRRENDMSPSNNVVPIHVPPTTENKPKVQCCQNI

SEQ ID NO: 3 - DNA, 696 bp, Homo sapiens
caatggggacccgggacgacgagtacgactacctattcaaagtggtgctcatcggggactcaggcgtgggcaagagcaacctgctgtcgcgcttcacccgcaacgagttcaacctggagagcaagagcaccatcggcgtggagttcgccacccgcagcatccaggtggacggcaagaccatcaaggcgcagatctgggacaccgctggccaggagcgctaccgcgccatcacctccgcgtactaccgtggtgcagtgggcgccctgctggtgtacgacatcgccaagcacctgacctatgagaacgtggagcgctggctgaaggagctgcgggaccacgcagacagcaacatcgtcatcatgctggtgggcaacaagagtgacctgcgccacctgcgggctgtgcccactgacgaggcccgcgccttcgcagaaaagaacaacttgtccttcatcgagacctcagccttggattccactaacgtagaggaagcattcaagaacatcctcacagagatctaccgcatcgtgtcacagaaacagatcgcagaccgtgctgcccacgacgagtccccggggaacaacgtggtggacatcagcgtgccgcccaccacggacggacagaagcccaacaagctgcagtgctgccagaacctgtgacccctgcgcctccacccagcgtgcgtgcacgtcctcc

SEQ ID NO: 4 - protein, 218 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNNLSFIETSALDSTNVEEAFKNILTEIYRIVSQKQIADRAAHDESPGNNVVDISVPPTTDGQKPNKLQCCQNL

SEQ ID NO: 5 - DNA, 903 bp, Mus musculus
ggctcgtcaccgggtccggcagctgaagctcctcgctcgctcgggttacccctgcagcgacgccccctggtcccgccgccgttgccaccgccgctcccgcccctcagctcctcggccgcgccatgggcacccgcgacgacgagtacgactacctctttaaagttgtccttattggagattctggtgttggaaagagtaacctcctgtctcgatttactcgaaatgagtttaatctggaaagcaagagtaccattggagtagagtttgcaacaagaagcatccaggttgatgggaaaacaataaaggcacagatatgggacacagcagggcaggagcggtacagggctataacgtctgcatactatcgtggagcagtaggtgccttattggtttatgacattgctaagcatctcacatatgaaaatgtagagcgatggctgaaagaactgagagatcatgctgatagtaacattgttatcatgcttgtgggcaataagagtgatttacgtcatctcagggcagttcctacagatgaagcaagagcttttgcagagaagaatggtttgtcattcattgagacatctgctctagattctacaaatgttgaagctgcttttcagacaattctaacagagatataccgcattgtttctcagaagcaaatgtcagacagacgtgaaaatgacatgtctccaagcaacaatgtggttcctattcatgttccgcccaccactgagaacaagccaaaggtgcagtgctgtcagaacatctaaggcgtctcttcccctagaaggctgtgtatagtccatttcccaggtctgagatttaaatatatttgtaattcttgtggtcaaaaaaaaaaaaaaaaaaaaaaaaaaaaaaaaaaaaaaaaaaaaaaaaaaa

SEQ ID NO: 6 - protein, 216 aa, Mus musculus; identical to SEQ ID NO: 2

SEQ ID NO: 7 - DNA, 657 bp, Mus musculus
atggggacccgggacgacgagtacgattacctattcaaagtggtgcttattggggactcaggtgtaggtaagagcaacctgctgtcacgcttcaccagaaacgaattcaacctagagagcaagagtaccatcggagtggagttcgccactcgcagcattcaggtggacggcaagaccatcaaggctcagatctgggacactgctggccaggagcgctaccgtgccattacctctgcgtactaccgtggtgcagtgggtgcactgctggtatatgacattgccaagcacttgacatatgagaacgtggagcgctggctgaaggagctgcgggatcatgcagatagcaacattgtcatcatgctggtgggcaacaagagtgacctgcgccaccttcgggctgtgcccactgatgaggcccgtgcctttgcagaaaagaacaacttgtccttcattgagacctcagccttggattccaccaatgtagaggaagcattcaagaacatcctcacagaaatctaccgtattgtgtcacagaagcaaatcgctgaccgtgcagcccacgatgagtcccctggcaacaacgtggtggacatcagtgtgccacccaccaccgatggacagagacccaacaagctgcagtgctgccagagcctgtga

SEQ ID NO: 8 - protein, 218 aa, Mus musculus [sequence omitted]

SEQ ID NO: 9 - DNA, 1401 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcagccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 10 - protein, 466 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 11 - DNA, 1665 bp, Homo sapiens
cgcgcgctcaggctcagcttcgctgcccgcccaggtagtgcccgctggagctcgcgcgctcatccggcaccactccagggctccaggctcctcgggcttccggagtcgagacgtggtggagttggctcgggctgaacttctttcggggggctgcctgtccttctttttgcagaagatgaatccggcctcggcgccccctccgctcccgccgcctgggcagcaagtgatccacgtcacgcaggacctagacacagacctcgaagccctcttcaactctgtcatgaatccgaagcctagctcgtggcggaagaagatcctgccggagtctttctttaaggagcctgattcgggctcgcactcgcgccagtccagcaccgactcgtcgggcggccacccggggcctcgactggctgggggtgcccagcatgtccgctcgcactcgtcgcccgcgtccctgcagctgggcaccggcgcgggtgctgcgggtagccccgcgcagcagcacgcgcacctccgccagcagtcctacgacgtgaccgacgagctgccactgcccccgggctgggagatgaccttcacggccactggccagaggtacttcctcaatcacatagaaaaaatcaccacatggcaagaccctaggaaggcgatgaatcagcctctgaatcatatgaacctccaccctgccgtcagttccacaccagtgcctcagaggtccatggcagtatcccagccaaatctcgtgatgaatcaccaacaccagcagcagatggcccccagtaccctgagccagcagaaccaccccactcagaacccacccgcagggctcatgagtatgcccaatgcgctgaccactcagcagcagcagcagcagaaactgcggcttcagagaatccagatggagagagaaaggattcgaatgcgccaagaggagctcatgaggcaggaagctgccctctgtcgacagctccccatggaagctgagactcttgccccagttcaggctgctgtcaacccacccacgatgaccccagacatgagatccatcactaataatagctcagatcctttcctcaatggagggccatatcattcgagggagcagagcactgacagtggcctggggttagggtgctacagtgtccccacaactccggaggacttcctcagcaatgtggatgagatggatacaggagaaaacgcaggacaaacacccatgaacatcaatccccaacagacccgtttccctgatttccttgactgtcttccaggaacaaacgttgacttaggaactttggaatctgaagacctgatccccctcttcaatgatgtagagtctgctctgaacaaaagtgagccctttctaacctggctgtaatcactaccattgtaacttggatgtagccatgaccttacatttcctgggcctcttggaaaaagtgatggagcagagcaagtctgcaggtgcaccacttcccgcctccatgactcgtgctccctcctttttatgttgccagtttaatcattgcctggttttgattgagagtaacttaagttaaacataaataaatattctattttcattttcaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - protein, 400 aa, Homo sapiens
MNPASAPPPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGSPAQQHAHLRQQSYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQSTDSGLGLGCYSVPTTPEDFLSNVDEMDTGENAGQTPMNINPQQTRFPDFLDCLPGTNVDLGTLESEDLIPLFNDVESALNKSEPFLTWL

SEQ ID NO: 13 - DNA, 2569 bp, Mus musculus
cggacgcgtggggccaaagtttctgtctcagttgggacgccgccgcggccgggggcaaagaaagggaggaaggaaggagctcgcggaggggaggggaggagaggggaggcggcctcgggcaaggagtgcagggcgatgcgggcgcgcgtcgcagccccccgaacctgagcgcagtgccccgagcgtcgaacgaggccgcagccatggagcccgcgcaacagccgccgccccagccggccccgcaaggccccgcgccgccgtccgtgtctccggccgggacccccgcggccccgcccgcacccccggccggccaccaggtcgtgcacgtccgcggggactcggagaccgacttggaggcgctcttcaatgccgtcatgaaccccaagacggccaacgtgcctcagaccgtgcccatgcggcttcgcaagctgcccgactccttcttcaagccgcctgagcccaagtcccactcgcgacaggccagtactgatgcaggtactgcgggagctctgactccacagcatgttcgagctcactcctctccagcctccctgcagctgggtgccgtttctcctgggacactcacagccagtggcgttgtctctggccctgccgctgcccctgcagctcagcatctccggcagtcctcctttgagatccctgatgatgtaccactgccagcaggctgggagatggccaagacatcttctggtcaaagatacttcttaaatcacaacgatcagacaacaacatggcaggacccccggaaggccatgctttcgcaactgaacgttcctgcgcctgccagcccagcggtgccccagacgctgatgaattctgcctcaggacctcttcctgatggatgggagcaagccatgactcaggatggagaagtttactacataaaccataagaacaagaccacatcctggctggacccaaggctggaccctcgttttgccatgaaccagaggatcactcagagtgctccagtgaagcagcccccacccttggctccccagagcccacagggaggcgtcctgggtggaggcagttccaaccagcagcagcaaatacagctgcagcagttacagatggagaaggagagactgcggttgaaacaacaggaattatttcggcaggcaatacggaatatcaatcccagcacagcaaatgctccaaaatgtcaggaattagctctgcgcagccagttgcctacactggagcaggatggagggactccgaatgcagtgtcttctcctgggatgtctcaggaattgagaacaatgacaaccaatagttccgatccctttcttaacagtggcacctatcactctcgagatgagagcacagacagcggcctcagcatgagcagctacagcatccctcggaccccagacgacttcctcaacagtgtggatgaaatggatacaggagacaccatcagccaaagcaccctgccgtcacagcagagccgcttccccgactacctggaagccctccctgggacaaatgtggaccttggcacactggaaggagatgcaatgaacatagaaggggaggagctgatgcccagtctgcaggaagcgctgagttccgaaatcttggacgtggagtctgtgttggctgccaccaagctagataaagaaagctttctcacgtggttatagagctgcagggagccactctgagtctgtgagggatccacagagcctaagatgtgcacgcctgaaattcagataagtcagtgggggttctctggctaacacagaaaacagatgaaccagtgtccatcgttgttccgcttttctctgcccgtcgctgctcttacgttggttgctgacctcttcacggccggctctaaagaacccgaaccgcagacagattcctttgttaactctgctatgataactacgttctctgggattgctgggggatggcctgctggataatggatgttctgccttttgtccggtggtcctttcaccatcactttaactgaacacacagactgggaactgaatgctctagaacattgttcaagaggtggtttcttcagctgccttgggtccaacaagccagaggcattgcgtctgatctcgtggaggacggaggggacccacgctgaagactggtgaactttccattcttctgttagcgatgccgttaggccatagtgacctgggtcttatcttagacgcttatgaggcatgagacagcttccatagaaatatattaattattaccacatactctagattaggtttgaatgaatattttctgtgggtgttttggttggtttttctctgcccccccccccttttttgtggttggtcttggtggaacgtaggcaaattaatgaattcgtttatagctgtagcttggggtgggcaataccattcttttggtgggaaatctgtatttcttggttttttaacatcctatttaaatcttaaatcttggttatctcctctctacatatatacacactcttattatgtctatggtagtgtgatagcagaatatatctttataaacaaaaaaaaaaaaaaaaaaaaaaaaaaaaaaa

SEQ ID NO: 14 - protein, 488 aa, Mus musculus
MEPAQQPPPQPAPQGPAPPSVSPAGTPAAPPAPPAGHQVVHVRGDSETDLEALFNAVMNPKTANVPQTVPMRLRKLPDSFFKPPEPKSHSRQASTDAGTAGALTPQHVRAHSSPASLQLGAVSPGTLTASGVVSGPAAAPAAQHLRQSSFEIPDDVPLPAGWEMAKTSSGQRYFLNHNDQTTTWQDPRKAMLSQLNVPAPASPAVPQTLMNSASGPLPDGWEQAMTQDGEVYYINHKNKTTSWLDPRLDPRFAMNQRITQSAPVKQPPPLAPQSPQGGVLGGGSSNQQQQIQLQQLQMEKERLRLKQQELFRQAIRNINPSTANAPKCQELALRSQLPTLEQDGGTPNAVSSPGMSQELRTMTTNSSDPFLNSGTYHSRDESTDSGLSMSSYSIPRTPDDFLNSVDEMDTGDTISQSTLPSQQSRFPDYLEALPGTNVDLGTLEGDAMNIEGEELMPSLQEALSSEILDVESVLAATKLDKESFLTWL

SEQ ID NO: 15 - DNA, 1610 bp, Mus musculus
gtccgggagccgcggcggctgcgctcgtctacgtcttctctgtcgcctcctcgcgcagtgggagcgcccgaggccggttccggggatgtaagaggataagccttcggctgctgggaatccgctcgggatctgcccgggaccgggttccagctcgtcagttcgggaggcgcccaggcttggcttccccgagtccccagaaagatgaatccgtcctcggtgccccatccgctcccgccgccagggcagcaagtcatccacgtcacgcaggacctggacaccgacctcgaagccctcttcaactctgtcatgaaccccaagcccagctcatggcggaaaaagatcctcccggagtccttctttaaggagcccgattccggctcgcactcgcgccaatccagcacagactcatcaggcggccacccggggcctcgactagctggcggcgcgcagcacgtccgctcgcactcgtcgcccgcatccctgcagctgggcaccggtgcgggagccgctggaggccctgcacagcagcatgcacatctccgccagcagtcctatgacgtgaccgacgagctgccgttgccccccgggtgggagatgaccttcacggccactggccagagatacttccttaatcacatagagaaaatcaccacatggcaagaccccaggaaggtgatgaatcagcctctgaatcatgtgaacctccacccgtccatcacttccacctcggtgccacagaggtccatggcagtgtcccagccgaatctcgcaatgaatcaccaacaccagcaagtcgtggccactagcctgagtccacagaaccacccgactcagaaccaacccacagggctcatgagtgtgcccaatgcactgaccactcagcagcagcagcagcagaaactgcggcttcagaggatccagatggagagagagaggattaggatgcgtcaagaggagctcatgaggcaggaagctgccctctgccgacagctccccatggaaaccgagaccatggcccctgtcaacacgcctgccatgagcacagatatgagatctgtcaccaacagtagctcagatcctttcctcaatggagggccctatcattcacgggagcagagcacagacagtggcctggggttagggtgctacagtgtccccacaactccagaagacttcctcagcaacatggacgagatggatacaggtgaaaattccggtcagacacccatgaccgtcaatccccagcagacccgcttccctgatttcctggactgccttccaggaacaaatgttgacctcgggactttggagtctgaagatctgatccctctcttcaatgatgtagagtctgctctgaacaaaagcgagccctttctaacctggctgtaatcactactgttgtaacgtgatgcagctgtgagctgacgcgcgtcttgggccttgcggaccaagtgatgaggcagagcgggcctgcagctgcaccacgttctgcctttgtactcacactccttgtccgtgtggccacttaatcattgcctggtgttgattcgcaggaacttgcgttacacagaaataaatactctcttttcattttcaaaaaaaaaaaaaaa

SEQ ID NO: 16 - protein, 395 aa, Mus musculus
MNPSSVPHPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGGPAQQHAHLRQQSYDVTDELPLPPGWEMTFTATGQRYFLNHIEKITTWQDPRKVMNQPLNHVNLHPSITSTSVPQRSMAVSQPNLAMNHQHQQVVATSLSPQNHPTQNQPTGLMSVPNALTTQQQQQQKLRLQRIQMERERIRMRQEELMRQEAALCRQLPMETETMAPVNTPAMSTDMRSVTNSSSDPFLNGGPYHSREQSTDSGLGLGCYSVPTTPEDFLSNMDEMDTGENSGQTPMTVNPQQTRFPDFLDCLPGTNVDLGTLESEDLIPLFNDVESALNKSEPFLTWL

(19) United States
(12) Patent Application Publication
(10) Pub. No.: US 2017/0114382 A1
(43) Pub. Date: Apr. 27, 2017

(54) METHODS OF INCREASING PROTEIN PRODUCTION IN MAMMALIAN CELLS

(71) Applicant: BIOGEN MA INC., Cambridge, MA (US)

(72) Inventors: John Follit, Leominster, MA (US); Scott Estes, Framingham, MA (US)

(73) Assignee: Biogen MA Inc., Cambridge, MA (US)

(21) Appl. No.: 15/115,323

(22) PCT Filed: Jan. 30, 2015
(86) PCT No.: PCT/US2015/013982

§ 371 Date: Jul. 29, 2016

Related U.S. Application Data
(60) Provisional application No. 61/934,661, filed on Jan. 31, 2014.

(51) Int. Cl. C12P 21/02; C12N 5/071; C12N 15/85; C12P 21/00

(52) U.S. Cl. CPC C12P 21/02; C12P 21/005; C12N 5/0682; C12N 15/85; C12N 2840/203

(57) ABSTRACT

Aspects of the present disclosure provide compositions and methods for increasing protein production in mammalian cells, e.g. methods of increasing mammalian cell expression of a protein of interest, comprising culturing mammalian cells that overexpress a protein of interest and are modified to overexpress a gene encoding Rab 11 or Yap1, as well as mammalian cells that overexpress a protein of interest and which are modified to overexpress a gene encoding Rab 11 or Yap1.

### RELATED APPLICATION

This application claims the benefit under 35 U.S.C. §119(e) of U.S. provisional application No. 61/934,661, filed Jan. 31, 2014, which is incorporated by reference herein in its entirety.

### FIELD OF THE INVENTION

Aspects of the present disclosure are in the field of mammalian cell protein production and, in some embodiments, relate particularly to mammalian cell production of therapeutic proteins.

### BACKGROUND

Mammalian cells, such as Chinese hamster ovary (CHO) cells, are typically used in the biopharmaceutical industry for the production of therapeutic proteins. These cells have an array of post-translational modifications, grow robustly and can thrive in suspension culture. Nonetheless, mammalian cells are not equipped to produce high levels of recombinant protein.

### SUMMARY OF THE INVENTION

The present disclosure is based, in part, on an improvement of host cell protein productivity that can be achieved through overexpression of particular genes that control cell secretion and cell size. Mammalian cells, such as CHO cells, are not professional secretory cells and, thus, are ill-equipped to handle the increased secretory flux required to produce high levels of recombinant protein. Results provided herein show that certain proteins of the Rab family, when overexpressed in mammalian cells, increase relevant metrics of titer and specific productivity. Without being bound by theory, it is believed that this improvement in mammalian cell protein productivity results from increased secretory capacity imparted by the overexpression of certain Rab proteins (e.g., Rab11). The present disclosure also shows that cellular overexpression of certain transcription factors of the Hippo pathway (e.g., Yap1), which controls cell proliferation, produces results similar to those observed with overexpression of certain Rab family proteins.

Thus, aspects of the present disclosure provide methods of increasing expression of a protein, comprising culturing mammalian cells that overexpress a protein of interest, wherein the cells are modified to overexpress a gene encoding Rab11 protein in addition to overexpressing the protein of interest. Some aspects of the present disclosure provide methods that comprise culturing mammalian cells that comprise a recombinant nucleic acid encoding a protein of interest and are modified to overexpress Rab11 protein. In some embodiments, cells are cultured in cell culture media under conditions that permit production and secretion of the protein of interest into the media. In some embodiments, methods further comprise isolating and/or purifying the protein of interest from the media.

Some aspects of the present disclosure provide mammalian cells that overexpress a protein of interest, wherein the cell is modified to overexpress a gene encoding Rab11 protein in addition to overexpressing the protein of interest. Some aspects of the present disclosure provide mammalian cells that comprise a recombinant nucleic acid encoding a protein of interest and are modified to overexpress Rab11 protein.

Some aspects of the present disclosure provide methods of producing modified mammalian cells, comprising modifying mammalian cells to express a Rab11 protein, and introducing into the mammalian cells a recombinant nucleic acid encoding a protein of interest, thereby producing engineered mammalian cells. In some embodiments, the methods further comprise culturing modified mammalian cells in media under conditions that permit production and secretion of the protein of interest into the media. In some embodiments, methods further comprise isolating and/or purifying the protein of interest from the media. In some embodiments, the step of modifying mammalian cells comprises introducing into the mammalian cells a recombinant nucleic acid encoding a Rab11 protein.

Thus, in some embodiments, mammalian cells comprise a recombinant nucleic acid encoding Rab11 protein. In some embodiments, a recombinant nucleic acid encoding Rab11 protein is expressed episomally. In some embodiments, a recombinant nucleic acid encoding Rab11 protein is expressed genomically.

In some embodiments, a recombinant nucleic acid encoding Rab11 and a recombinant nucleic acid encoding a protein of interest are expressed from the same vector (e.g., a DNA molecule used as a vehicle to carry genetic material into another cell). In some embodiments, a recombinant nucleic acid encoding Rab11 and a recombinant nucleic acid encoding a protein of interest are expressed from the same plasmid (e.g., capable of independent replication).

In some embodiments, a Rab11 protein is stably expressed in mammalian cells. In some embodiments, a protein of interest is stably expressed in mammalian cells.

In some embodiments, mammalian cells are Chinese hamster ovary (CHO) cells.

In some embodiments, a Rab11 protein is a Rab11a isoform or a Rab11b isoform. In some embodiments, a Rab11 protein is a Rab11b isoform.

In some embodiments, a protein of interest is a therapeutic protein. In some embodiments, a therapeutic protein is an antibody. For example, an antibody may be a monoclonal antibody.

Some aspects of the present disclosure provide methods of increasing expression of a protein, comprising culturing mammalian cells that overexpress a protein of interest, wherein the cells are modified to overexpress a gene encoding Yap1 and/or Taz protein in addition to overexpressing the protein of interest. Some aspects of the present disclosure provide methods that comprise culturing mammalian cells that comprise a recombinant nucleic acid encoding a protein of interest and are modified to overexpress Yap1 and/or Taz protein. In some embodiments, cells are cultured in cell culture media under conditions that permit production and secretion of the protein of interest into the media. In some embodiments, methods further comprise isolating and/or purifying the protein of interest from the media.

Some aspects of the present disclosure provide mammalian cells that overexpress a protein of interest, wherein the cell is modified to overexpress a gene encoding Yap1 and/or Taz protein in addition to overexpressing the protein of interest. Some aspects of the present disclosure provide mammalian cells that comprise a recombinant nucleic acid encoding a protein of interest and are modified to overexpress Yap1 and/or Taz protein.

Some aspects of the present disclosure provide methods of producing modified mammalian cells, comprising modifying mammalian cells to express a Yap1 and/or Taz protein, and introducing into the mammalian cells a recombinant nucleic acid encoding a protein of interest, thereby producing engineered mammalian cells. In some embodiments, the methods further comprise culturing modified mammalian cells in media under conditions that permit production and secretion of the protein of interest into the media. In some embodiments, methods further comprise isolating and/or purifying the protein of interest from the media. In some embodiments, the step of modifying mammalian cells comprises introducing into the mammalian cells a recombinant nucleic acid encoding a Yap1 and/or Taz protein.

Thus, in some embodiments, mammalian cells comprise a recombinant nucleic acid encoding Yap1 and/or Taz protein.

Thus, in some embodiments, mammalian cells comprise a recombinant nucleic acid encoding Yap1 and/or Taz protein. In some embodiments, a recombinant nucleic acid encoding Yap1 and/or Taz protein is expressed episomally. In some embodiments, a recombinant nucleic acid encoding Yap1 and/or Taz protein is expressed genomically.

In some embodiments, a recombinant nucleic acid encoding Yap1 and/or Taz and a recombinant nucleic acid encoding a protein of interest are expressed from the same vector (e.g., a DNA molecule used as a vehicle to carry genetic material into another cell). In some embodiments, a recombinant nucleic acid encoding Yap1 and/or Taz and a recombinant nucleic acid encoding a protein of interest are expressed from the same plasmid (e.g., capable of independent replication).

In some embodiments, a Yap1 and/or Taz protein is stably expressed in mammalian cells. In some embodiments, a protein of interest is stably expressed in mammalian cells.

In some embodiments, mammalian cells are Chinese hamster ovary (CHO) cells.

In some embodiments, a protein of interest is a therapeutic protein. In some embodiments, a therapeutic protein is an antibody. For example, an antibody may be a monoclonal antibody.

### BRIEF DESCRIPTION OF THE DRAWINGS

FIG. 1A shows a graph of cell specific productivity (qP) data obtained from an analysis of DG44i Chinese hamster ovary (CHO) cells modified to stably express Rab11b or Yap1 protein and to produce an antibody of interest; FIG. 1B shows a graph of antibody titer data produced with the modified CHO cells;

FIG. 2A shows a graph of antibody titer data obtained from an analysis of the top five clones originating from DG44i CHO cells modified to stably express Rab11b or Yap1 protein and to produce an antibody of interest; FIG. 2B shows a graph of antibody titer data obtained from an analysis of the top 24 clones originating from CHO cells modified to stably express Rab11b or Yap1 protein and to produce monoclonal antibody;

FIG. 3A shows a graph of specific productivity data obtained form an analysis of the top five clones originating from DG44i CHO cells modified to stably express Rab11b or Yap1 protein and to produce an antibody of interest; FIG. 3B shows a graph of specific productivity data obtained from an analysis of the top 24 clones originating from DG44i CHO cells modified to stably express Rab11b or Yap1 protein and to produce monoclonal antibody;

FIG. 4 shows a graph of data obtained from an antibody titer analysis of CHO-S cells modified to stably express Rab11b or Yap1 protein and to produce an antibody of interest;

FIG. 5 shows a graph of specific productivity data obtained from an analysis of CHO-S cells modified to stably express Rab11b or Yap1 protein and to produce an antibody of interest;

FIG. 6, left panel, shows a graph of antibody titer data obtained from a primary screen of unamplified Rab11b cell lines expressing a monoclonal antibody of interest (v. DG44 control); FIG. 6, right top panel, shows a graph of antibody titer data obtained from an analysis of top Rab11b amplified mini-pools (v. DG44 control); FIG. 6, right bottom panel, shows a graph of specific productivity data obtained from an analysis of top Rab11b amplified mini-pools (v. DG44 control);

FIG. 7 shows a graph of antibody titer data obtained from a primary screen of amplified and enriched Rab11b cell lines (v. DG44 control);

FIG. 8A shows a graph of antibody titer data obtained from an analysis of top Yap1 amplified mini-pools (v. DG44 control); FIG. 8B shows a graph of specific productivity data obtained from an analysis of top Yap1 amplified mini-pools (v. DG44 control);

FIG. 9 shows a graph of antibody titer data obtained from a primary screen analysis of amplified and enriched Yap1 cell lines (v. DG44 control);

FIG. 10A shows a graph of antibody titer data obtained from an analysis of amplified and enriched Yap1 cell lines (v. DG44 control); FIG. 10B shows a graph of specific productivity data obtained from an analysis of amplified and enriched Yap1 cell lines (v. DG44 control);

FIGS. 11A-11C show graphs of data obtained from assessments of the product quality of recombinant protein expressed from engineered Rab11b, Yap1 and DG44 host cell lines; protein aggregation (FIG. 11A), product related impurity profiling (FIG. 11B) and glycan analysis (FIG. 11C) were assessed;

FIG. 12A shows a graph of antibody titer data obtained from an analysis of the top overall cell line from Rab11b and Yap1 host cell lines (v. DG44 control); and FIG. 12B shows a graph of antibody titer data obtained using a third antibody of interest (e.g. mAb3).

### DETAILED DESCRIPTION OF THE INVENTION

The production of recombinant proteins, such as therapeutic proteins (e.g., antibodies), places high demands on the secretory capacity of mammalian cells due to the fact that such cells are not “professional” secretory cells (e.g., cells capable of secreting thousands of proteins per second). That is, mammalian cells do not contain a highly developed endoplasmic reticulum, where newly synthesized proteins must fold and assemble to native structures before secretion. Consequently, mammalian cells are not equipped to handle the increased secretory flux required of a cell to produce high levels of recombinant protein. The present disclosure is based, in part, on the surprising increase in secretory capacity and/or cell size of a mammalian cell that results from the overexpression of certain individual genes and that can increase relevant metrics of titer and cell specific productivity (qP). Thus, aspects of the present disclosure provide compositions and methods for increasing protein production in mammalian cells through overexpression of certain regulatory genes. As used herein, “overexpression” refers to expression of a gene or protein in a modified cell at a level greater than a level of expression of the same gene or protein in an unmodified cell. Surprisingly, overexpression of certain regulatory genes, as provided herein, increases the ability of a cell to secrete one or more recombinant proteins without adversely affecting intracellular synthesis, sorting and trafficking of recombinant proteins of interest. “Regulatory genes,” as used herein, refers to genes encoding proteins that regulate, or contribute to the regulation of, a cell function (e.g., cell secretion, cell proliferation).

In some embodiments, a protein is considered to be overexpressed in a modified cell if the expression level of the protein is at least 10%, at least 20%, at least 30%, at least 40% or at least 50% greater than the expression level of the same protein in an unmodified cell. For example, the expression level of an overexpressed protein may be 10%, 15%, 20%, 25%, 30%, 35%, 40%, 45%, 50%, 60%, 65%, 70%, 75%, 80%, 85%, 90%, 95%, 100%, 200% greater than the expression level of the same protein in an unmodified cell. In some embodiments, a protein is considered to be overexpressed in a modified cell if the expression level of the protein is (or is at least) 10% to 200%, 10% to 100%, 10% to 50%, 20% to 200%, 20% to 100%, or 20% to 50% greater than the expression level of the same protein in an unmodified cell.

Regulatory genes provided herein include those that encode members of the Rab family of proteins, which is one of five main families in the Ras superfamily of monomeric G proteins. Rab proteins regulate vesicular transport pathways in exocytic and endocytic pathways, for example, regulating the movement of membrane vesicles between intra-cellular compartments. There are approximately 70 different Rab proteins that have been identified in humans and most are involved primarily in vesicle trafficking.

Some aspects of the present disclosure relate to overexpression of Rab11 proteins. Rab11 is known to associate primarily with perinuclear recycling endosomes and regulates recycling of endocytosed proteins (Takahashi S., et al. 2012 J. Cell Sci. 125, 4049-4057). There are at least two known human isoforms of Rab11, including Rab11a (NCBI Ref. No. NC_000015.9; NCBI Accession Nos. BC013348 (SEQ ID NO: 1) and AAH13348 (SEQ ID NO: 2)) and Rab11b (NCBI Ref. No. NC_000019.9; NCBI Accession Nos. BC110081 (SEQ ID NO: 3) and AAI10082 (SEQ ID NO: 4)). Similarly, there are at least two known mouse isoforms of Rab11, including Rab11a (NCBI Ref. No. NC_000075.6; NCBI Accession Nos. BC010722 (SEQ ID NO: 5) and AAH10722 (SEQ ID NO: 6)) and Rab11b (NCBI Ref. No. NC_000083.6; NCBI Accession Nos. AB232606 (SEQ ID NO: 7) and BAF02868 (SEQ ID NO: 8)). In some embodiments, a human Rab protein (e.g., human Rab11a or human Rab11b) is overexpressed in mammalian cells, and thus mammalian cells that express recombinant human Rab protein are provided herein. In other embodiments, a mouse Rab protein (e.g., mouse Rab11a or mouse Rab11b) is overexpressed in mammalian cells, and thus mammalian cells that express recombinant mouse Rab protein are provided herein. Additional aspects of the present disclosure provide mammalian cells that comprise nucleic acids encoding human or mouse Rab proteins. Further, in some embodiments, a host cell Rab11 protein is overexpressed. For example, an endogenous human Rab11 protein may be overexpressed in a human cell, an endogenous mouse Rab11 protein may be overexpressed in a mouse cell, an endogenous Chinese hamster Rab11 protein may be overexpressed in a Chinese hamster cell (e.g., a CHO cell), or other endogenous Rab11 proteins may be overexpressed in other cells.

It should be appreciated that a heterologous (e.g., from a different species, such as a different mammalian species) Rab11 protein can be overexpressed in a mammalian cell line being used to overexpress a protein of interest.

In some embodiments, the cell productivity of mammalian cells that overexpress Rab11 and a protein of interest is at least 5% greater than the cell specific productivity of mammalian cells that are not modified to comprise a nucleic acid encoding a Rab11 protein. In some embodiments, cell productivity of mammalian cells that overexpress Rab11 and a protein of interest is (or is at least) 5%, 10%, 15%, 20%, 25%, 30%, 35%, 40%, 45%, 50%, 55%, 60%, 65%, 70%, 75%, 80%, 85%, 90%, 95%, or 100% greater than the cell specific productivity of mammalian cells that are not modified to comprise a nucleic acid encoding a Rab11 protein. In some embodiments, cell productivity of mammalian cells that overexpress Rab11 and a protein of interest is (or is at least) 10 to 100%, 10 to 50%, 20 to 100%, or 20 to 50% greater than the cell specific productivity of mammalian cells that are not modified to comprise a nucleic acid encoding a Rab11 protein.

Regulatory genes provided herein also include those that encode members of the Hippo signaling pathway, also referred to as the Salvador/Warts/Hippo (SWH) pathway. This pathway controls organ size in animals through the regulation of cell proliferation and apoptosis. Transcriptional coactivators of the Hippo signaling pathway include Yes-associated protein (YAP) and transcriptional coactivator with PDZ-binding motif (TAZ) (Wang K., et al. 2009 Biochemistry and Cell Biology 87 (1): 77-91), which bind to the transcription factor, Scalloped (Sd) in its active, unphosphorylated form to activate expression of transcriptional targets that promote cell growth, cell proliferation, and prevent apoptosis.

Some aspects of the present disclosure relate to the overexpression of proteins of the Hippo signaling pathway such as, for example, Yap1 and Taz. In some embodiments, a human Yap1 protein (NCBI Ref. No. NC_000011.9; NCBI Accession Nos. AB567720 (SEQ ID NO: 9) and BAJ41471 (SEQ ID NO: 10)) and/or a human Taz protein (NCBI Ref. No. NC_000003.11; NCBI Accession Nos. AJ299431.1 (SEQ ID NO: 11) and CAC17722.1 (SEQ ID NO: 12)) is overexpressed in mammalian cells, and thus mammalian cells that express recombinant human Yap1 protein and/or a human Taz protein are provided herein. In other embodiments, a mouse Yap1 protein (NCBI Ref. No. NC_000075.6; NCBI Accession Nos. BC014733 (SEQ ID NO: 13) and AAH14733 (SEQ ID NO: 14)) and/or a mouse Taz protein (NCBI Ref. No. NC_000069.6; NCBI Accession Nos. BC004640 (SEQ ID NO: 15) and AAH04640 (SEQ ID NO: 16)) is overexpressed in mammalian cells, and thus mammalian cells that express recombinant mouse Yap1 protein and/or a human Taz protein are provided herein. Additional aspects of the present disclosure provide mammalian cells that comprise nucleic acids encoding human or mouse Yap1 and/or Taz proteins. Further, in some embodiments, a host cell Yap1 and/or Taz protein is overexpressed. For example, an endogenous human Yap1 and/or Taz protein may be overexpressed in a human cell, an endogenous mouse Yap1 and/or Taz protein may be overexpressed in a mouse cell, an endogenous Chinese hamster Yap1 and/or Taz protein may be overexpressed in a Chinese hamster cell (e.g., a CHO cell), or other endogenous Yap1 and/or Taz proteins may be overexpressed in other cells.

It should be appreciated that a heterologous (e.g., from a different species, such as a different mammalian species) Yap1 protein and/or Taz protein can be overexpressed in a mammalian cell line being used to overexpress a protein of interest.

In some embodiments, the cell productivity of mammalian cells that overexpress Yap1 and/or Taz and a protein of interest is (or is at least) 5% greater than the cell specific productivity of mammalian cells that are not modified to comprise a nucleic acid encoding a Yap1 and/or Taz protein. In some embodiments, mammalian cells that overexpress Yap1 and/or Taz and a protein of interest is (or is at least) 10%, 15%, 20%, 25%, 30%, 35%, 40%, 45%, 50%, 55%, 60%, 65%, 70%, 75%, 80%, 85%, 90%, 95%, or 100% greater than the cell specific productivity of mammalian cells that are not modified to comprise a nucleic acid encoding a Yap1 and/or Taz protein.

Accordingly, some aspects of the present disclosure relate to overexpression of one or more regulatory proteins in mammalian cells. Mammalian cells include, for example, human cells, primate cells, rodent cells (e.g., mouse and rat cells), and canine cells. Mammalian cells lines for use in accordance with the present disclosure include, without limitation, 293-T, 3T3 cells, 4T1, 721, 9L, A-549, A172, A20, A253, A2780, A2780ADR, A2780cis, A431, ALC, B16, B35, BCP-1 cells, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, HEK-293, HeLa, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, Ma-Mel 2, Ma-Mel 3 . . . Ma-Mel 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT cell lines, Peer, PNT-1A/PNT 2, PTK2, Raji, RBL cells, RenCa, RIN-5F, RMA/RMAS, S2, Saos-2 cells, SiHa, SKBR3, SKOV-3, T-47D, T2, T84, THP1 cell line, U373, U87, U937, VCaP, Vero cells, WM39, WT-49, X63, YAC-1 and YAR cells.

In some embodiments, Chinese hamster ovary (CHO) cells are used in accordance with the present disclosure. Any CHO cell line may be used, as provided herein. Examples of CHO cell lines include, without limitation, DG44 cells, DUXB11 cells, CHOK1 cells, and CHO-S cells.

As used herein, a “modified cell” refers to a cell that contains a nucleic acid that is not present in an unmodified cell. In some embodiments, a modified cell contains a mutation in a genomic nucleic acid. In some embodiments, a modified cell contains an independently replicating nucleic acid that is not present in an unmodified nucleic acid. In some embodiments, a modified cell is produced by introducing a foreign or exogenous nucleic acid into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), or microinjection of purified DNA directly into the nucleus of the cell (see, e.g., Capecchi M. R. Cell. 1980 November; 22(2 Pt 2): 479-88).

A vector (e.g., plasmid) may include an origin of replication and, optionally, a selectable marker.

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing a recombinant nucleic acid into the cell in order to produce a genetic change of interest (e.g., via insertion or homologous recombination)

In some embodiments, a nucleic acid that is introduced into a cell encodes a regulatory protein (e.g., Rab11, Yap1, and/or Taz) operably connected to a promoter and/or other transcriptional control element. In some embodiments, a nucleic acid that is introduced into a cell provides a promoter and/or transcriptional control element (e.g., enhancer) that can be used to increase expression of an endogenous regulatory protein (e.g., an endogenous Rab11, Yap1 and/or Taz), for example, via homologous recombination or insertion at or near the endogenous gene encoding the regulatory protein.

In some embodiments, a regulatory protein (e.g., Rab11, Yap1 and/or Taz protein) is constitutively overexpressed in a modified mammalian cell. In some embodiments, a regulatory protein (e.g., Rab11, Yap1 and/or Taz protein) is under the control of an inducible promoter in a modified mammalian cell.

In some embodiments, a mammalian cell also can be modified to express a protein of interest (e.g., a therapeutic protein). That is, a modified cell as provided herein may comprise a deoxyribonucleic acid (DNA) that is transcribed to messenger ribonucleic acid (mRNA), which is then translated into polypeptide chains, which are ultimately folded into proteins. In some embodiments, a protein of interest is transiently expressed in a cell, while in other embodiments, a protein of interest is stably expressed in a cell. Accordingly, in some embodiments a cell that overexpresses a regulatory protein (e.g., Rab11, Yap1 and/or Taz) is modified to express a protein of interest. In some embodiments, a cell is modified to overexpress both the regulatory protein and the protein of interest. In some embodiments, a modified cell contains recombinant genes that encode a regulatory protein (e.g., a Rab11, Yap1 and/or Taz protein) and a protein of interest. In some embodiments, the recombinant genes are under the control of an inducible promoter. In some embodiments, the regulatory protein(s) and the protein(s) of interest are under the control of the same inducible promoter. In some embodiments, the regulatory protein(s) and the protein(s) of interest are under the control of different inducible promoters. In some embodiments, one or both the regulatory protein(s) and the protein(s) of interest are transiently expressed. In some embodiments, one or both the regulatory protein(s) and the protein(s) of interest are stably expressed.

“Transient cell expression” herein refers to expression by a cell of a nucleic acid that is not integrated into the nuclear genome of the cell. By comparison, “stable cell expression” herein refers to expression by a cell of a nucleic acid that remains in the nuclear genome of the cell and its daughter cells. Typically, to achieve stable cell expression, a cell is co-transfected with a marker gene and an exogenous nucleic acid that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. In some embodiments, Geneticin, also known as G418, is used as an agent for selecting stable transfection of mammalian cells. This toxin can be neutralized by the product of the neomycin resistance gene. Other marker genes/selection agents are contemplated herein. Examples of such marker genes and selection agents include, without limitation, dihydrofolate reductase with methotrexate, glutamine synthetase with methionine sulphoximine, hygromycin with hygromycin phosphotransferase, and puromycin with puromycin n acetyltransferase

Mammalian cells engineered to comprise a nucleic acid (e.g., a nucleic acid encoding a protein of interest) may be cultured using conventional mammalian cell culture methods (see, e.g., Phelan M. C. Curr Protoc Cell Biol. 2007 September; Chapter 1: Unit 1.1).

In some embodiments, culture media used as provided herein may be commercially available and/or well-described (see, e.g., Birch J. R., R. G. Spier (Ed.) Encyclopedia of Cell Technology, Wiley. 2000, 411-424; Keen M. J. Cytotechnology 1995; 17: 125-132; Zang, et al. Bio/Technology. 1995; 13: 389-392).

In some aspects, mammalian cells may be cultured to a density of about 1×10⁴ to 1×10⁸ viable cells/ml cell culture media. In some embodiments, cells are cultured to a density of about 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸ viable cells/ml. In some embodiments, cells are cultured to a density of about 2×10⁵ to 3×10⁷ viable cells/ml.

In some aspects, mammalian cells are cultured in a bioreactor. A bioreactor refers to a container in which cells are cultured, for example, a culture flask, dish, or bag that may be single-use (disposable), autoclavable, or sterilizable. The bioreactor may be made of glass, or it may be polymer-based, or it may be made of other materials. In some embodiments, a bioreactor is made of linear low-density polyethylene (LLDPE), for example, a LLDPE WAVE Bioreactor™ (GE Healthcare™).

In some embodiments, a bioreactor refers to a cell culture bioreactor, including a stirred tank (e.g., well-mixed) bioreactor or tubular reactor (e.g., plug flow), airlift bioreactor, membrane stirred tank, spin filter stirred tank, vibromixer, fluidized bed reactor, or a membrane bioreactor. The mode of operating a bioreactor may be a batch or continuous processes and will depend on the cell strain being cultured. A bioreactor is continuous when the feed and product streams are continuously being fed and withdrawn from the system. A batch bioreactor may have a continuous recirculating flow, but no continuous feeding of nutrient or product harvest. For intermittent-harvest and fed batch (or batch fed) cultures, cells may be inoculated at a lower viable cell density in a medium that is similar in composition to a batch medium. Cells may be allowed to grow exponentially with essentially no external manipulation until nutrients are somewhat depleted and cells are approaching stationary growth phase. At this point, for an intermittent harvest batch-fed process, a portion of the cells and product may be harvested, and the removed culture medium is replenished with fresh medium. This process may be repeated several times. For production of proteins of interest (e.g., fusion proteins, antibodies), a fed batch process may be used. While cells are growing exponentially, but nutrients are becoming depleted, concentrated feed medium (e.g., 10-15 times concentrated basal medium) maybe added either continuously or intermittently to supply additional nutrients, allowing for further increase in cell concentration and the length of the production phase. Fresh medium may be added proportionally to cell concentration without removal of culture medium (broth). To accommodate the addition of medium, a fed batch culture may be started in a volume much lower that the full capacity of the bioreactor (e.g., approximately 40% to 50% of the maximum volume).

In some embodiments, cells are cultured using a perfusion-based high cell density seed train expansion procedure, involving the creation of a high cell density cell bank. The high density cell bank vials are used to directly inoculate a seed train bioreactor, for example, a perfusion WAVE Bioreactor™ (GE Healthcare™) (see, e.g., Tao et al. Biotechnol Prog. 2011; 00(00): 1-6 (published online)).

In some embodiments, methods comprise isolating and/or purifying a protein of interest from cell culture media or a cell preparation that contains Rab11, or Yap1 and Taz (e.g., Rab11, Yap1 and/or Taz produced recombinantly). Purification refers, generally, to the process by which a protein of interest (e.g., therapeutic antibody) is separated from non-protein components of a mixture. Protein purification methods are known in the art, any of which may be used in accordance with the present disclosure. Non-limiting examples of protein purification methods include size exclusion chromatography, separation based on charge or hydrophobicity, affinity chromatography, and high-performance liquid chromatography. Purified protein may also be concentrated by, for example, ultrafiltration. In some embodiments, proteins of interest (e.g., obtained from a cell preparation that contains Rab11/Yap1/Taz) are lyophilized.

Also provided herein are crude cell preparations comprising a protein of interest and trace amounts of Rab11, or Yap1 and/Taz (e.g., Rab11, or Yap1 and/or Taz produced recombinantly). A “trace amount” of a protein may be an amount that is 5% or less (or less than 5%) of the preparation. In some embodiments, a trace amount of a protein is 0.001% to 5%. In some embodiments, a trace amount of a protein is 0.001% to 0.01%, 0.001% to 0.1%, or 0.01% to 0.1%.

Some aspects of the present disclosure relate to cells engineered to comprise nucleic acids, for example, encoding one or more proteins of interest or other proteins, as provided herein. As used herein, the term “nucleic acid” refers to at least two nucleotides covalently linked together, and in some instances, may contain phosphodiester bonds (e.g., a phosphodiester “backbone”). Nucleic acids (e.g., components, or portions, of the nucleic acids) of the present disclosure may be naturally occurring or engineered. Engineered nucleic acids include recombinant nucleic acids and synthetic nucleic acids. “Recombinant nucleic acids” refer to molecules that are constructed by joining nucleic acid molecules (e.g., naturally-occurring or synthetic) and, in some embodiments, can replicate in a living cell. “Synthetic nucleic acids” refer to molecules that are chemically, or by other means, synthesized or amplified, including those that are chemically or otherwise modified but can base pair with naturally occurring nucleic acid molecules. Recombinant and synthetic nucleic acids also include those molecules that result from the replication of either of the foregoing.

Nucleic acids may be single-stranded (ss) or double-stranded (ds), as specified, or may contain portions of both single-stranded and double-stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xanthine, hypoxanthine, isocytosine, and isoguanine.

In some embodiments, a nucleic acid comprises a promoter sequence, or promoter, operably linked to a nucleotide sequence encoding a protein of interest. As used herein, a “promoter” refers to a control region of a nucleic acid sequence at which initiation and rate of transcription of the remainder of a nucleic acid sequence are controlled. A promoter may also contain subregions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be classified as strong or weak according to its affinity for RNA polymerase (and/or sigma factor); this is related to how closely the promoter sequence resembles the ideal consensus sequence for the polymerase. The strength of a promoter may depend on whether initiation of transcription occurs at that promoter with high or low frequency. Different promoters with different strengths may be used to construct genetic circuits with different levels of gene/protein expression (e.g., the level of expression initiated from a weak promoter is lower than the level of expression initiated from a strong promoter).

A promoter may be one naturally associated with a gene or sequence, as may be obtained by isolating the 5′ non-coding sequences located upstream of the coding segment and/or exon of a given gene or sequence. Such a promoter can be referred to as “endogenous.” Similarly, an enhancer may be one naturally associated with a nucleic acid sequence, located either downstream or upstream of that sequence.

In some embodiments, a coding nucleic acid segment may be positioned under the control of a recombinant or heterologous promoter, which refers to a promoter that is not normally associated with the encoded nucleic acid sequence in its natural environment. A recombinant or heterologous enhancer refers to an enhancer not normally associated with a nucleic acid sequence in its natural environment. Such promoters or enhancers may include promoters or enhancers of other genes; promoters or enhancers isolated from any other prokaryotic cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. No. 4,683,202 and U.S. Pat. No. 5,928,906).

As used herein, an “inducible promoter” is one that is characterized by initiating or enhancing transcriptional activity when in the presence of, influenced by or contacted by an inducer or inducing agent. An “inducer” or “inducing agent” may be endogenous or a normally exogenous condition, compound or protein that contacts a genetic circuit in such a way as to be active in inducing transcriptional activity from the inducible promoter.

In some embodiments, a promoter may or may not be used in conjunction with an “enhancer,” which refers to a cis-acting regulatory sequence involved in the transcriptional activation of a nucleic acid sequence downstream of the promoter. The enhancer may be located at any functional location before or after the promoter.

In some embodiments, a mammalian cell is engineered to overexpress a regulatory protein (e.g., Rab11, Yap1 and/or Taz) and also comprise a nucleic acid that encodes a protein of interest. As used herein, a “protein of interest” refers to any protein that is encoded by a nucleic acid and can be expressed in a mammalian cell. It should be appreciated that a protein of interest may be, for example, monomeric, homomultimeric or hetermultimeric. Thus, in some embodiments, multiple genes, under the same promoter or under different promoters, may be introduced into a cell to encode multiple polypeptide chains of a protein of interest. In some embodiments, a protein of interest is a recombinant protein. A “recombinant protein” herein refers to a protein encoded by a recombinant nucleic acid.

In some embodiments, a protein of interest is a therapeutic protein. Therapeutic proteins can be divided into groups, as follows (a) proteins that replace a protein that is deficient or abnormal; (b) proteins that augment an existing pathway; (c) proteins that provide a novel function or activity; (d) proteins that interfere with a molecule or organism; and (e) proteins that deliver (e.g., are conjugated to) other compounds or proteins, such as a radionuclide, cytotoxic drug, or effector proteins. Therapeutic proteins can also be grouped based on their molecular types that include antibody-based drugs, Fc fusion proteins, anticoagulants, blood factors, bone morphogenetic proteins, engineered protein scaffolds, enzymes, growth factors, hormones, interferons, interleukins, and thrombolytics. Therapeutic proteins can also be classified based on their molecular mechanism of activity as (a) binding non-covalently to target, e.g., mAbs; (b) affecting covalent bonds, e.g., enzymes; and (c) exerting activity without specific interactions, e.g., serum albumin. In some embodiments, a therapeutic protein is a recombinant therapeutic protein.

In some embodiments, provided herein are mammalian cells that overexpress Rab11, Yap1, and/or Taz, and that also comprise a nucleic acid that encodes a therapeutic protein. In some embodiments, provided herein are mammalian cells engineered to comprise a nucleic acid encoding a Rab11 protein and a nucleic acid encoding a therapeutic protein (e.g., antibody). In some embodiments, provided herein are mammalian cells engineered to comprise a nucleic acid encoding a Yap1 and/or Taz protein and a nucleic acid encoding a therapeutic protein (e.g., antibody).

Non-limiting examples of therapeutic proteins include insulin, growth hormone somatotropin, neuroblastin, tau, mecasermin, Factor VIII, Factor IX, antibthrombin III, Protein C, erythropoietin, filgrastin, sargramostin, oprelvekin, human follicle-stimulating hormone, interferon, collagenase, hyaluronidase, papain, L-asparaginase, peg-asparaginase, lepirudin, bivalirudin, streptokinase and anistreplase. Other therapeutic proteins are contemplated herein.

In some embodiments, a mammalian cell may be engineered to comprise a nucleic acid encoding an antibody or an antigen binding fragment thereof. As used herein, the term “antibody” refers to a Y-shaped protein used by the immune system to identify and neutralize foreign objects (e.g., bacteria and viruses). In some embodiments, an antibody may be a glycoprotein comprising at least two heavy (H) chains and two light (L) chains inter-connected by disulfide bonds. The term “antigen-binding fragment” of an antibody as used herein, refers to one or more portions of an antibody that retain the ability to specifically bind to an antigen. It has been shown that the antigen-binding function of an antibody can be performed by fragments of a full-length antibody.

The term “monoclonal antibody,” as used herein, refers to a preparation of antibody molecules of single molecular composition. A monoclonal antibody displays a single binding specificity and affinity for a particular epitope. In some embodiments, antibodies are chimeric or humanized antibodies. As used herein, the term “chimeric antibody” refers to an antibody that combines the murine variable or hypervariable regions with the human constant region or constant and variable framework regions. As used herein, the term “humanized antibody” refers to an antibody that retains only the antigen-binding CDRs from the parent antibody in association with human framework regions (see, e.g., Waldmann, Science 1991; 252: 1657). In some embodiments, antibodies are human antibodies. The term “human antibody,” as used herein, refers to an antibody having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). The term “human antibody,” as used herein, is not intended to include antibodies in which CDR sequences derived from the germline of another mammalian species, such as a mouse have been grafted onto human framework sequences (referred to herein as “humanized antibodies”). Antibodies provided herein encompass various antibody isotypes, such as IgG1, IgG2, IgG3, IgG4, IgM, IgA1, IgA2, IgAsec, IgD, IgE (Aase A et al. Eur J Immunol. 1993 July; 23(7):1546-51; Rijkers T et al. Infect. Immun. 1995, 63(1):73; Litvack M K et al. 2011 PLoS ONE 6(3): e17223; Weisbart R H et al. Nature. 1988 Apr. 14; 332(6165):647-8; Gorter A et al. Immunology. 1987 July; 61(3): 303-309; and Karagiannis S N et al. J Immuno 2007; 179:2832-2843). As used herein, “isotype” refers to the antibody class (e.g., IgM or IgG1) that is encoded by heavy chain constant region genes.

Examples of antibodies that may be produced by the methods described herein include 3F8, 8H9, abagovomab, abciximab, actoxumab, adalimumab, adecatumumab, aducanumab, afelimomab, afutuzumab, alacizumab pegol, ALD, alemtuzumab, alirocumab, altumomab pentetate, amatuximab, anatumomab mafenatox, anifrolumab, anrukinzumab (or IMA-638), apolizumab, arcitumomab, aselizumab, atinumab, atlizumab (or tocilizumab), atorolimumab, bapineuzumab, basiliximab, bavituximab, bectumomab, belimumab, benralizumab, bertilimumab, besilesomab, bevacizumab, bezlotoxumab, biciromab, bimagrumab, bivatuzumab mertansine, blinatumomab, blosozumab, brentuximab vedotin, briakinumab, brodalumab, canakinumab, cantuzumab mertansine, cantuzumab ravtansine, caplacizumab, capromab pendetide, carlumab, catumaxomab, cBR-doxorubicin immunoconjugate, cedelizumab, certolizumab pegol, cetuximab, citatuzumab bogatox, cixutumumab, clazakizumab, clenoliximab, clivatuzumab tetraxetan, conatumumab, concizumab, crenezumab, dacetuzumab, daclizumab, dalotuzumab, daratumumab, demcizumab, denosumab, detumomab, dorlimomab aritox, drozitumab, duligotumab, dupilumab, dusigitumab, ecromeximab, eculizumab, edobacomab, edrecolomab, efalizumab, efungumab, eldelumab, elotuzumab, elsilimomab, enavatuzumab, enlimomab pegol, enokizumab, enoticumab, ensituximab, epitumomab cituxetan, epratuzumab, erlizumab, ertumaxomab, etaracizumab, etrolizumab, evolocumab, exbivirumab, fanolesomab, faralimomab, farletuzumab, fasinumab, FBTA, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, guselkumab, ibalizumab, ibritumomab tiuxetan, icrucumab, igovomab, IMAB, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, ligelizumab, lintuzumab, lirilumab, lodelcizumab, lorvotuzumab mertansine, lucatumumab, lumiliximab, mapatumumab, margetuximab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-CD, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, ontuxizumab, oportuzumab monatox, oregovomab, orticumab, otelixizumab, otlertuzumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pinatuzumab vedotin, pintumomab, placulumab, polatuzumab vedotin, ponezumab, priliximab, pritoxaximab, pritumumab, PRO 140, quilizumab, racotumomab, radretumab, rafivirumab, ramucirumab, ranibizumab, raxibacumab, regavirumab, reslizumab, rilotumumab, rituximab, robatumumab, roledumab, romosozumab, rontalizumab, rovelizumab, ruplizumab, samalizumab, sarilumab, satumomab pendetide, secukinumab, seribantumab, setoxaximab, sevirumab, sibrotuzumab, SGN-CD19A, SGN-CD33A, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, teneliximab, teplizumab, teprotumumab, TGN, ticilimumab (or tremelimumab), tildrakizumab, tigatuzumab, TNX-650, tocilizumab (or atlizumab), toralizumab, tositumomab, tovetumab, tralokinumab, trastuzumab, TRBS, tregalizumab, tremelimumab, tucotuzumab celmoleukin, tuvirumab, ublituximab, urelumab, urtoxazumab, ustekinumab, vantictumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab and zolimomab aritox.

In some embodiments, an antibody produced by the methods and cells provided herein is an anti-lingo (e.g., anti-LINGO-1) antibody (see, e.g., U.S. Pat. No. 8,425,910). Anti-LINGO-1, for example, is a fully human monoclonal antibody that targets LINGO-1, a protein expressed selectively in the central nervous system (CNS) that is known to negatively regulate axonal myelination and axonal regeneration (Mi S, et al. Nat Neurosci. 2004; 7:221-8; Mi S, et al. Nat Neurosci. 2005; 8:745-51).

In some embodiments, an antibody produced by the methods and cells provided herein is an anti-amyloid BETA antibody. BART, for example, is a fully human IgG1 and was generated antibody. Anti-BART (e.g., BIIB037/aducanumab) is a human anti-amyloid BETA monoclonal antibody that was generated (Dunstan R, et al. Alzheimer's & Dementia: the journal of the Alzheimer's Association 2011, 7:S457).

In some embodiments, an antibody produced by the methods and cells provided herein is an anti-integrin αvβ5 antibody.

Other antibodies and therapeutic proteins of interest may be produced by methods and cells as provided herein.

Aspects of the invention are further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1

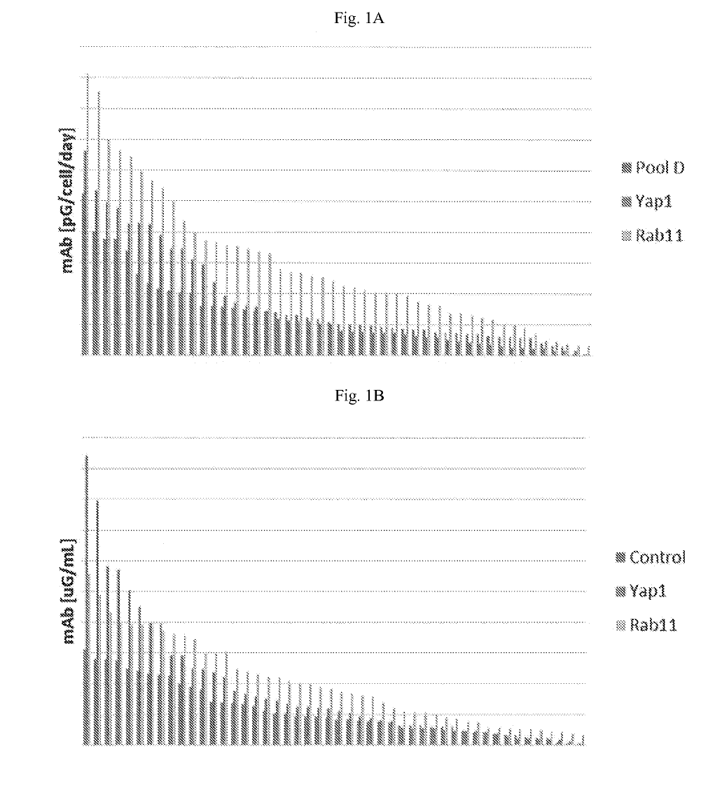

To determine if an increase in the secretory capacity of a Chinese hamster ovary (CHO) cell correlates with an increase in relative metrics of protein titer and specific productivity, DG44i host cells were engineered to express one of fifteen genes. The engineered CHO cells were evaluated with a model therapeutic antibody and examined at the uncloned pool stage. Several pools displayed increases in titer and specific productivity compared to unmodified DG44i (FIGS. 1A and 1B). Two of these pools were selected for further analysis at the clone stage; those modified by Yap1 and Rab11 expression.

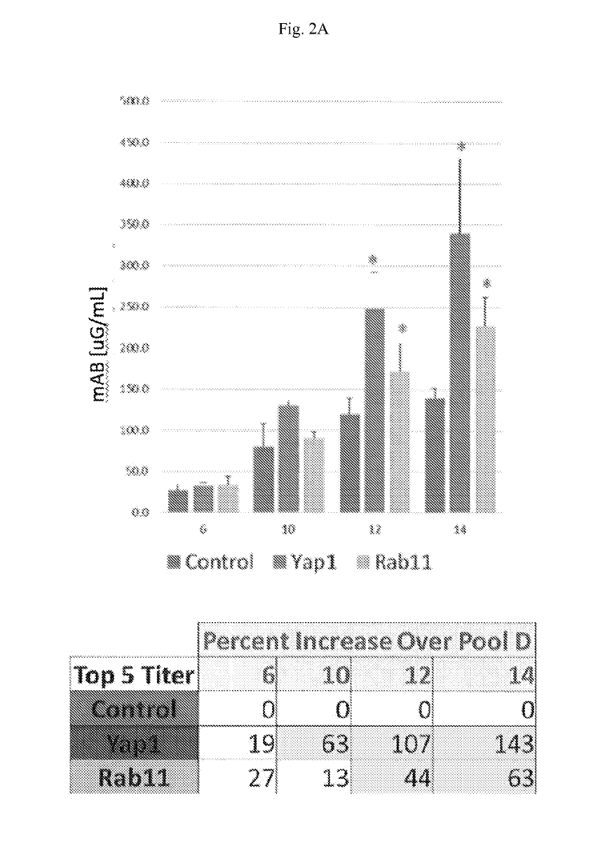

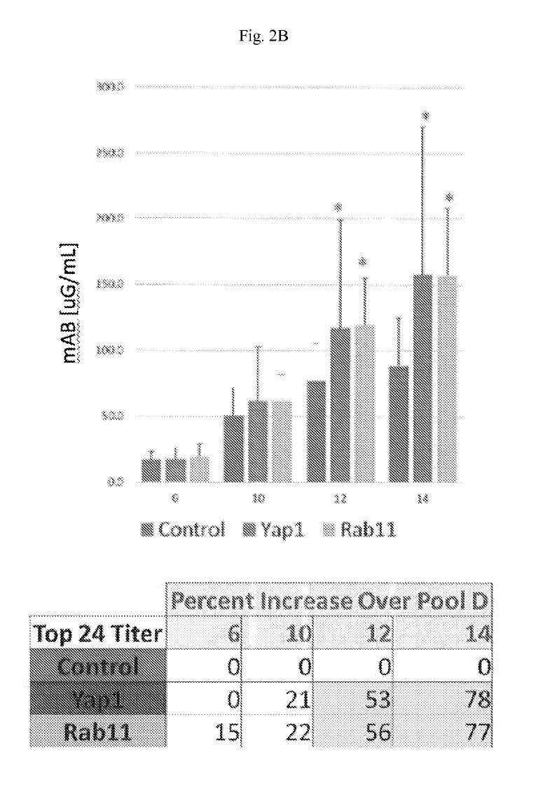

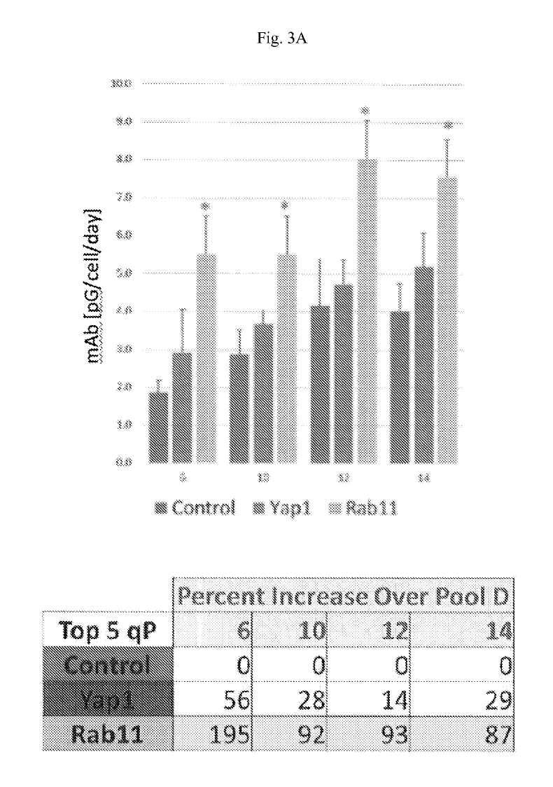

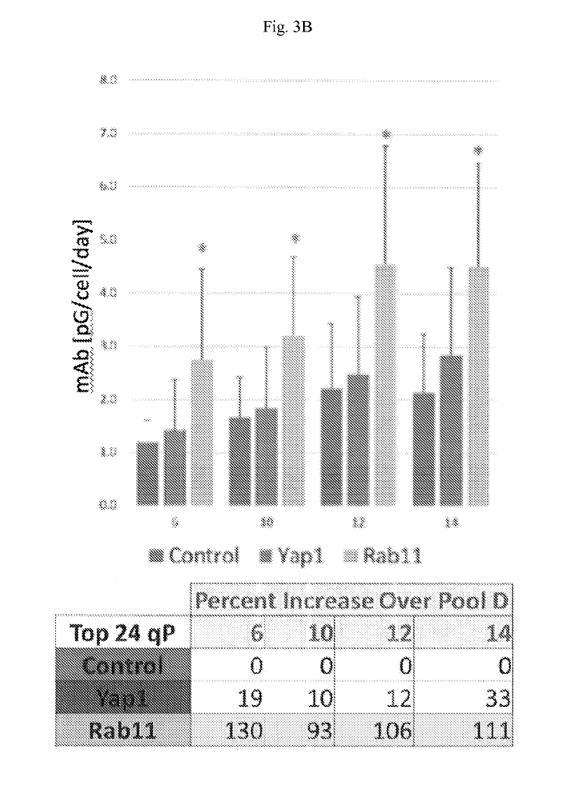

Rab11b and Yap1 were stably expressed in CHO cells. The engineered cells were then used to express a model therapeutic antibody. Forty-eight clones from each host were examined in a fed batch. Analysis of the top five clones originating from the engineered cell lines, Rab11b and Yap1, result in two-fold increases in specific productivity (FIGS. 3A and 3B) and titer (FIGS. 2A and 2B), respectively (p<0.05).

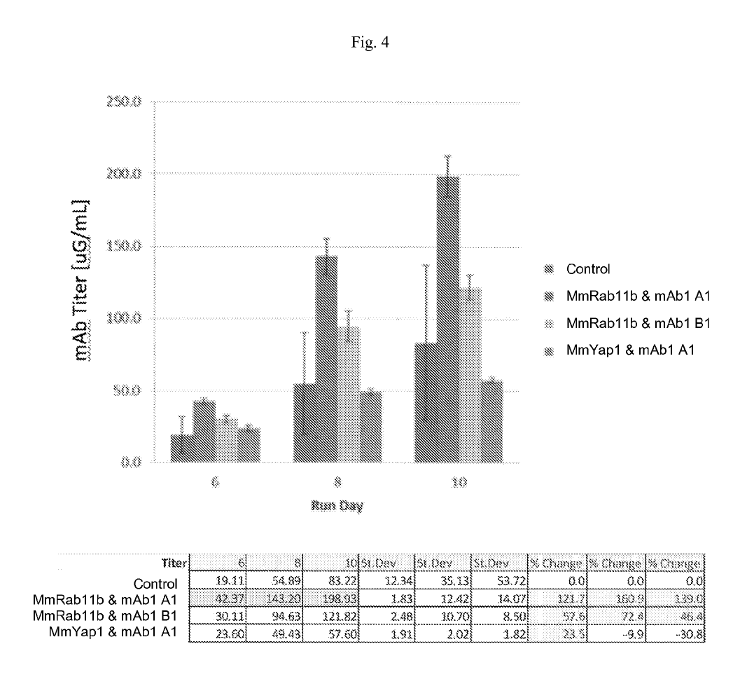

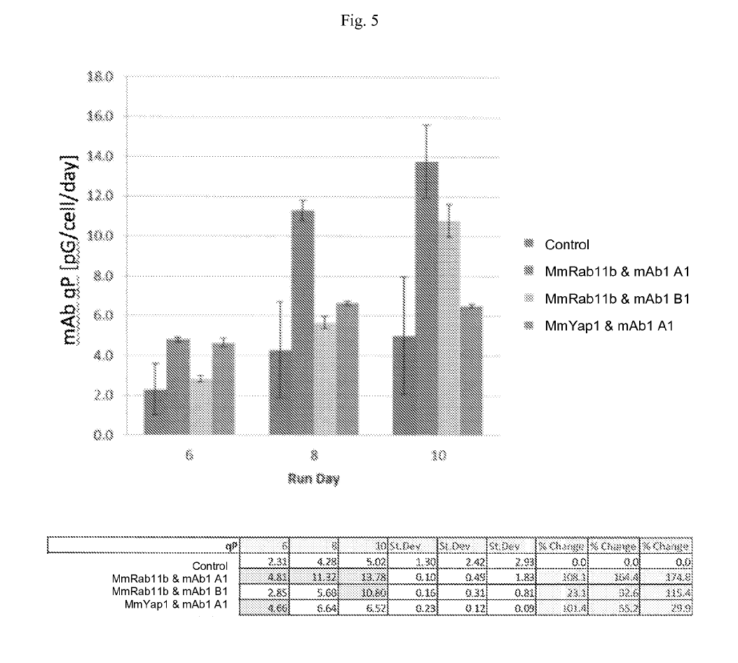

The expression of Rab11 and Yap1 was also examined in another host cell line, CHO-S. Data from CHO-S pools stably expressing Rab11b or Yap1 in combination with the model therapeutic antibody show similar increases in titer and productivity for the Rab11 pools (FIGS. 4 and 5) and an increase in productivity from the Yap1-derived pool (FIG. 5).

Materials & Methods Chinese Hamster Ovary (CHO) cells of the DG44 lineage were engineered to express myc/DDK tagged Rab11b or Yap1 using commercially obtained vectors from Origene (Cat#MR202439, MR226049). The DNA encoding Rab11b or Yap1 was introduced by electroporation and cells expressing the target genes were selected using G418. Target protein expression was confirmed via Western blot analysis on whole cell lysates from the recovered pools.

The Rab11 and Yap1 engineered pools were then auditioned with a model monoclonal antibody. DNA encoding the monoclonal antibody with an IRES linked dihydrofolate reductase selectable marker was introduced via electroporation to each of the engineered host lines and selected in nucleoside free media. The resulting pools were verified for target protein expression via Western blot and tested for mAb expression using an established Octet titer assay (ForteBio).

Clones were generated by limited dilution cloning from each of the pools derived from the engineered hosts (Rab11b & Yap1) and an unmodified DG44i control expressing the mAb. Briefly, cells were plated at 0.5 cell/well, expanded and 96 clones from each host were screened for mAb expression via Octet at the 96 well stage (primary screen).

The top 48 clones from each of the three hosts (Rab11b, Yap1, and control DG44) were then evaluated in a fourteen day fed batch process (secondary screen). The cells were seeded (Day 0), counted and fed on days (3, 6, 10, 12) and analyzed for titer on days (6, 10, 12, 14) using the Octet assay (ForteBio). Specific productivity (qP) and titer of the resulting clones were compared using a Student's T test and the percent increase in titer and qP of the engineered hosts was compared to controls (unmodified DG44).

### Example 2

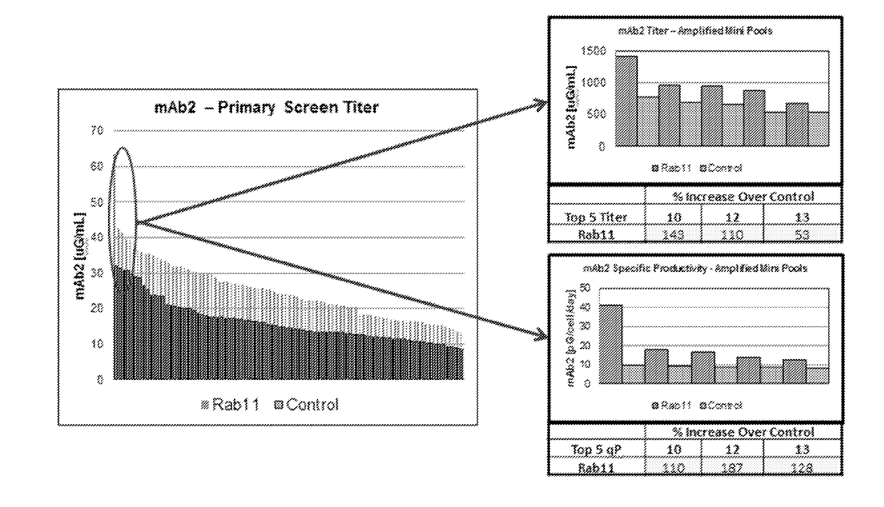

Experiments were next conducted to investigate whether the enhanced productivity seen with Rab11b and Yap1 overexpression was molecule specific or could be achieved with other molecules. To this end, host cell lines were auditioned with a second monoclonal antibody (mAb2). Stable cell lines expressing Rab11b, Yap1 or unmodified DG44 host cells were engineered to express mAb2. A primary screen of unamplified cell lines expressing mAb2 confirmed the positive benefits of Rab11b and Yap1 expression observed with mAb1 (FIG. 6, left panel, data not shown).

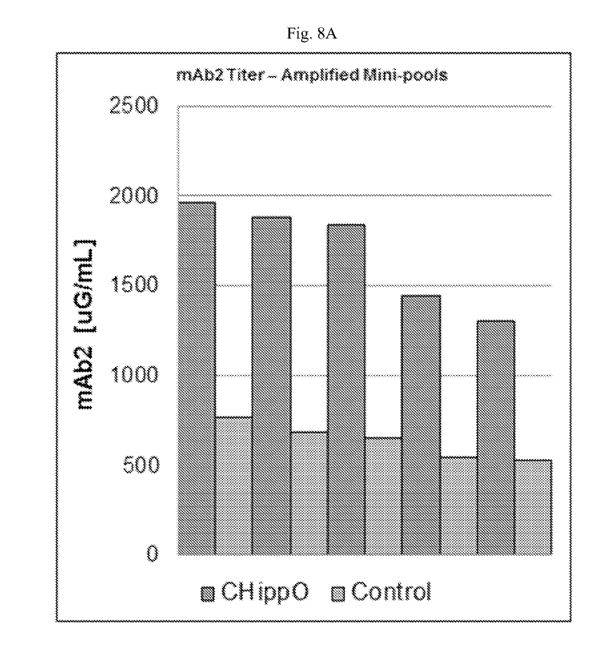

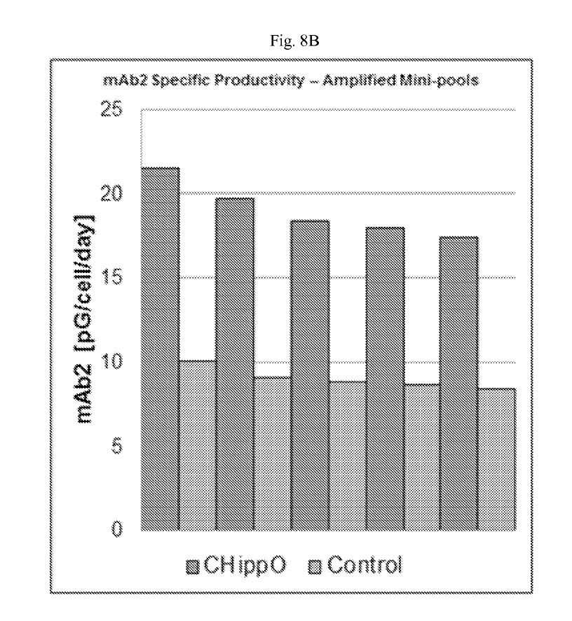

Next, to further increase the expression of mAb2, the top three unamplified cell lines from each of the engineered hosts (Rab11b & Yap1) and unmodified DG44 control were amplified with varying concentrations of methotrexate. Analysis of the top amplified mini-pools resulting from Rab11b and Yap1 hosts cell lines showed greater than two-fold increases in both titer (FIG. 6, top right panel, and FIG. 8A) and specific productivity (FIG. 6, bottom right panel, and FIG. 8B) compared to unmodified DG44.

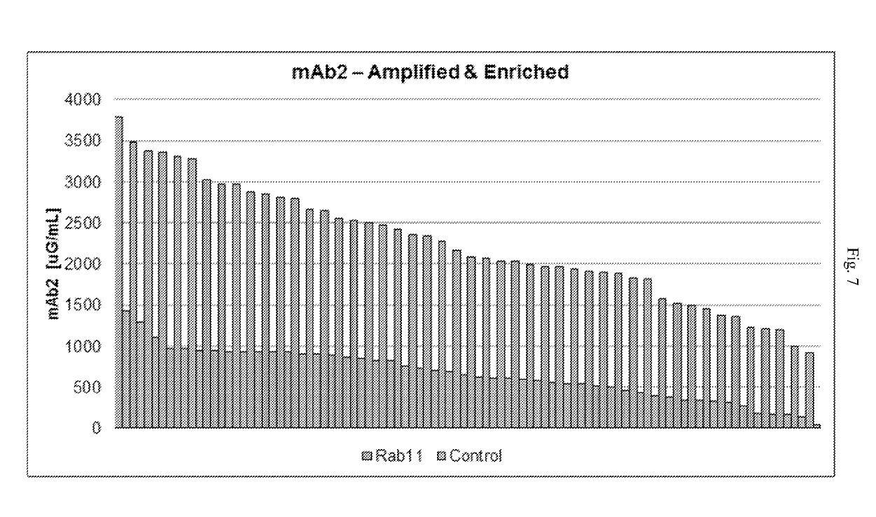

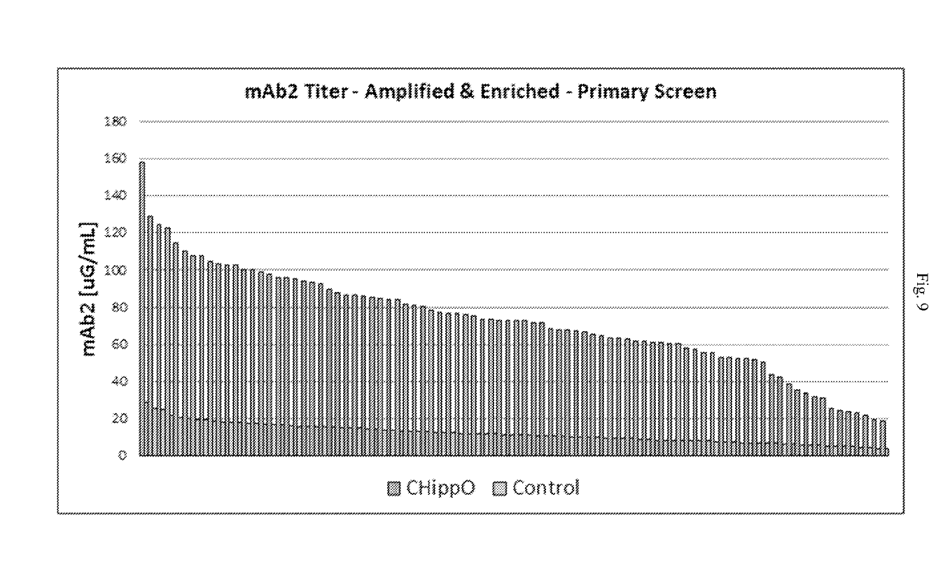

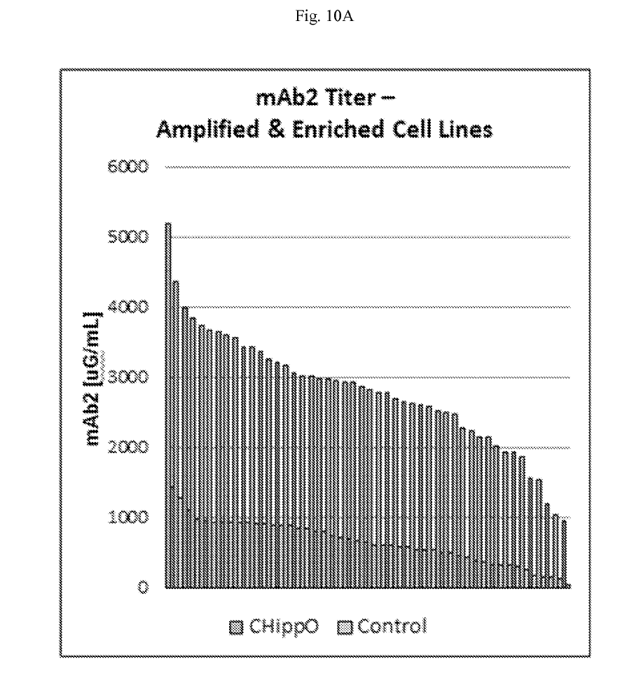

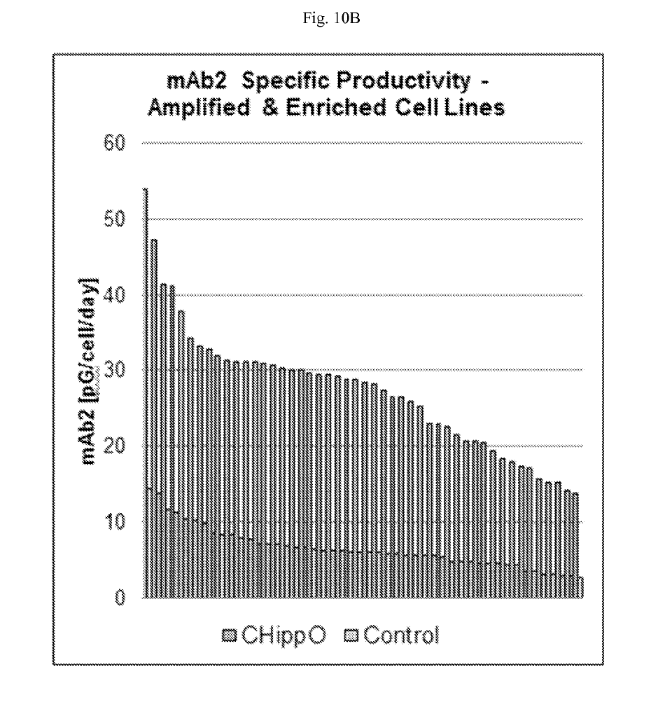

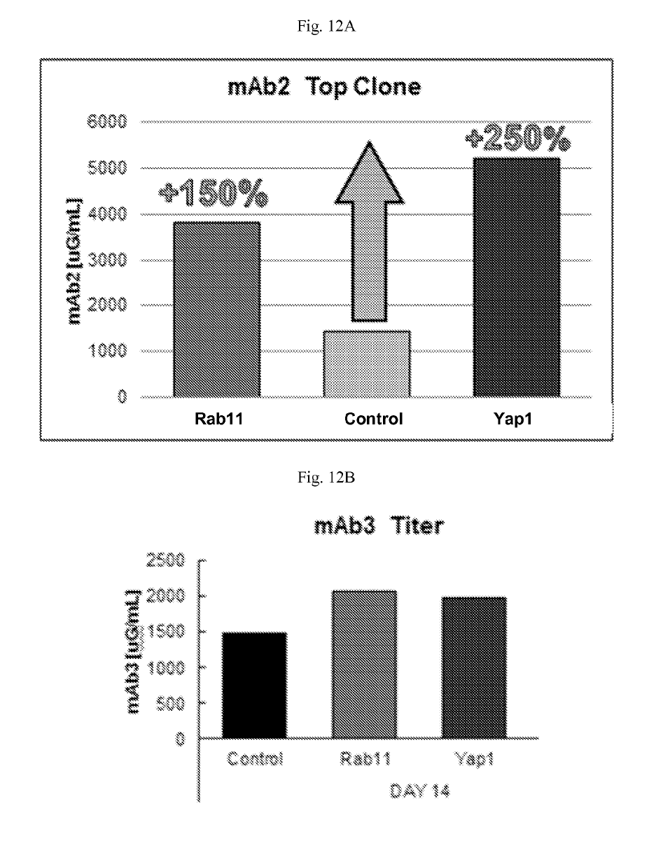

Finally, the top amplified mini-pools from Rab11b, Yap1 and control host lines were enriched using a ClonePixFL. Ninety-six of the resulting amplified and enriched cell lines from each host (Rab11b, Yap1 & DG44) were analyzed in a primary screen confirming the positive effects of both Rab11b and Yap1 expression during amplification and enrichment (FIGS. 7 and 9). Finally, the top forty-eight amplified and enriched cell lines from each host cell line were analyzed in a 14 day fed batch process (FIGS. 10A-10B, 12A). Cell lines derived from both the Rab11b and Yap1 engineered hosts showed significant increases (greater than 150%) in both titer (FIGS. 10A and 12A) and specific productivity (FIG. 10B) compared to unmodified DG44. These results in total confirm that the Rab11b and Yap1 engineering could enhance the expression of more than one molecule.

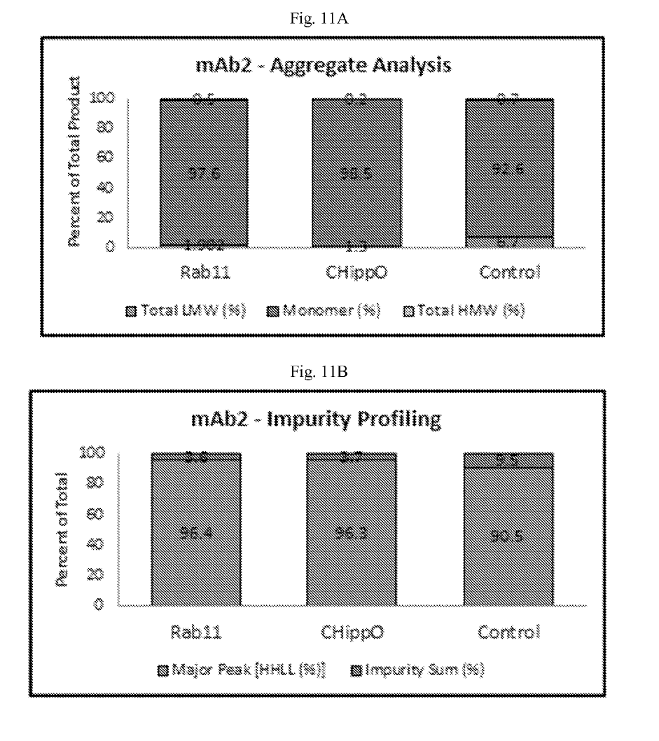

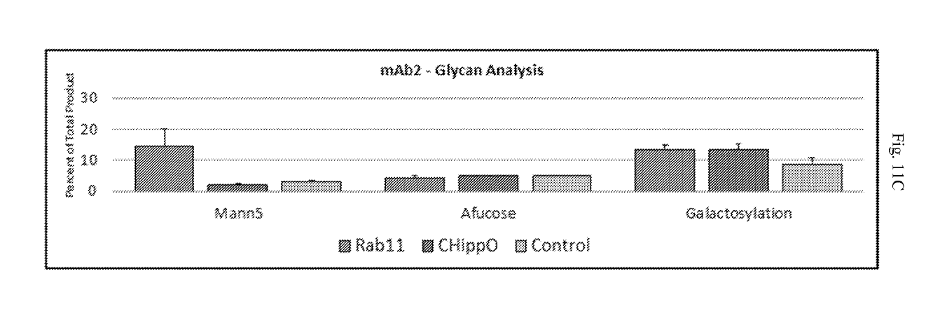

To assess the product quality of the recombinant protein expressed from these engineered hosts, mAb2 from the top five amplified and enriched cell lines from each of the three host lineages (Rab11b, Yap1 & DG44) was analyzed. Metrics assessed were, protein aggregation (FIG. 11A), product related impurity profiling (FIG. 11B) and glycan analysis (FIG. 11C). The results obtained showed that mAb2 expressed from either of the engineered host cell lines was essentially identical to that produced from the unmodified host with the exception of slightly elevated high mannose glycans found on mAb2 expressed from the Rab11b engineered host.

Materials and Methods.

Chinese Hamster Ovary (CHO) cells of the DG44 lineage were engineered to express myc/DDK tagged Rab11b or Yap1 via transfection with plasmid expressing the gene of interest off the hCMV promoter. The DNA encoding Rab11b or Yap1 was introduced by electroporation and cells expressing the target genes were selected using G418. Target protein expression was confirmed via Western blot analysis on whole cell lysates from the recovered pools.

The Rab11 and Yap1 engineered uncloned pools, along with the unmodified DG44 host were then auditioned with a model monoclonal antibody (mAb2). Following DNA electroporation, cells were plated at varying cell densities in 384 well plates and selected in nucleoside free media. The resulting cell lines were subjected to primary and secondary screens similar to mAb1 in Example 1.

The top mAb2 cell lines from each of the engineered (Rab11b & Yap1) and an unmodified DG44 were selected for amplification and enrichment. Briefly, the top three cell lines from each host were pooled and 100 cells/well were amplified in 384 well plates containing varying concentrations of methotrexate. Following primary and secondary screening of the resulting amplified mini-pools, the top mini-pool from each host cell line was selected for enrichment via the ClonePixFL (Molecular Devices) as outlined by the manufacturer. Cells lines selected by the ClonePix were subjected to a final primary and secondary screen as described above. The top producing amplified and enriched cell lines from Rab11b, Yap1 and unmodified DG44 were analyzed for key product quality attributes including aggregation (size exclusion chromatography), impurity profiling (capillary electrophoresis), and glycan analysis (high performance liquid chromatography).

SEQUENCES

SEQ ID NO: 1, RAB11A, Homo sapiens
gttgaagctc ggcgctcggg ttacccctgc agcgacgccc cctggtccca cagataccac
tgctgctccc gccctttcgc tcctcggccg cgcaatgggc acccgcgacg acgagtacga
ctacctcttt aaagttgtcc ttattggaga ttctggtgtt ggaaagagta atctcctgtc
tcgatttact cgaaatgagt ttaatctgga aagcaagagc accattggag tagagtttgc
aacaagaagc atccaggttg atggaaaaac aataaaggca cagatatggg acacagcagg
gcaagagcga tatcgagcta taacatcagc atattatcgt ggagctgtag gtgccttatt
ggtttatgac attgctaaac atctcacata tgaaaatgta gagcgatggc tgaaagaact
gagagatcat gctgatagta acattgttat catgcttgtg ggcaataaga gtgatctacg
tcatctcagg gcagttccta cagatgaagc aagagctttt gcagaaaaga atggtttgtc
attcattgaa acttcggccc tagactctac aaatgtagaa gctgcttttc agacaatttt
aacagagatt taccgcattg tttctcagaa gcaaatgtca gacagacgcg aaaatgacat
gtctccaagc aacaatgtgg ttcctattca tgttccacca accactgaaa acaagccaaa
ggtgcagtgc tgtcagaaca tctaaggcat ttctcttctc ccctagaagg ctgtgtatag
tccatttccc aggtctgaga tttaaatata tttgtaattc ttgtgtcact tttgtgtttt
attacttcat acttatgaat ttttccatgt cctaagtctt ttgattttag ctttataaaa
tcatccactt gtcccgaatg actgcagctt tttttcatgc tatggcttca ctagccttag
tttaataaac tgaatgtttg gattcctcag ttattgttta cttttcatca tggaagcctg
tcactgtatg taggacataa tagaacttga tcacttgaag ctcagaccta ttggtcttga
tcaaatcaaa ctaagaagac cttagaaata agctaccatt ttgccacaga gcagcttata
ggtaatacac tcttctctca gtgcagtgta catttccaca aatctaagaa ttgccctata
aacatagcag gattttgaga gcttgaaaat tttccattat tctggacatg aatttctaaa
atgccttaat aggtttatgt agttgagtaa attttgtttt ttaatttttg taagcatcaa
agttgattag agaggggggc actttttctg gagaattctc ttagtaaaca caaaagattg
ttacggtttc attagtagta tggttgtggg gccataagtt aaacagtgct gcctggtagg
ctgggaactg aagagacttg tggtattcca tctcgggtgc ctctgttggc aatgatcagg
cagcccaaaa gatttaaatg atctataata atttccaagc ggtagattat gtggcatttt
attgctcagg caataattgg tttaatgctg gtagtgtcaa attttgtctt agaaccttcc
agtaagtgaa atacaaccta gttttatcac catatccacc agcaggcatg gataattatt
ttaacaatgc taatatttga gttttgcagt atattataga atatagtcca gttaaatctt
tggtttcagt atgtctgaag agtacagtga gaggttaatt tctgctcaag tggtaccact
taaaggcatg tattctttta gtatgtaaaa tgaaatagta ccttgagttt aaatagaatg
catttaggca ttgtagagat ctgaaatagt tttcttccac tacattgttg aaatcaatga
agcaattagt ttctcattca gaaatgtgca cactaatatt tagttttgct ttctcgtgga
taatattaag cacttactct gcagtttcct ggaagttgtg tcaactgcag tgatactatt
caggatggtg ggaaatcccc aaaaatatgt atgtgtgggc ttgcttagat tactatattt
catagttaat cttttgtctc ttgcggtgct catgatgtgt ggggcacacg gaaggcattg
ctgtagtcag tcattttggt tttcttctat agccatttta ttattttagt gtattagtta
tgaagataat attatctatt tgtaaattgc tactttgtat tttatgcatg ctctgtaatt
tgattttttt ttagttattg atttggatta tattcacatt ctaataaaca gttatagggg
gaaaaaaaaa aaaaaaaaaa aaaaaaaaaa aaaaaaaaaa aaaaaaaaaa aaaaaaaaaa
aaaaaaaaaa aaaaaaaaaa aaaaaaaaaa aaaaaaaaaa aaaaaaaaaa aaaaaaaaaa
aaaaa
SEQ ID NO: 2, RAB11A, Homo sapiens
MGTRDDEYDY LFKVVLIGDS GVGKSNLLSR FTRNEFNLES KSTIGVEFAT RSIQVDGKTI
KAQIWDTAGQ ERYRAITSAY YRGAVGALLV YDIAKHLTYE NVERWLKELR DHADSNIVIM
LVGNKSDLRH LRAVPTDEAR AFAEKNGLSF IETSALDSTN VEAAFQTILT EIYRIVSQKQ
MSDRRENDMS PSNNVVPIHV PPTTENKPKV QCCQNI
SEQ ID NO: 3, RAB11B, Homo sapiens
caatggggac ccgggacgac gagtacgact acctattcaa agtggtgctc atcggggact
caggcgtggg caagagcaac ctgctgtcgc gcttcacccg caacgagttc aacctggaga
gcaagagcac catcggcgtg gagttcgcca cccgcagcat ccaggtggac ggcaagacca
tcaaggcgca gatctgggac accgctggcc aggagcgcta ccgcgccatc acctccgcgt
actaccgtgg tgcagtgggc gccctgctgg tgtacgacat cgccaagcac ctgacctatg
agaacgtgga gcgctggctg aaggagctgc gggaccacgc agacagcaac atcgtcatca
tgctggtggg caacaagagt gacctgcgcc acctgcgggc tgtgcccact gacgaggccc
gcgccttcgc agaaaagaac aacttgtcct tcatcgagac ctcagccttg gattccacta
acgtagagga agcattcaag aacatcctca cagagatcta ccgcatcgtg tcacagaaac
agatcgcaga ccgtgctgcc cacgacgagt ccccggggaa caacgtggtg gacatcagcg
tgccgcccac cacggacgga cagaagccca acaagctgca gtgctgccag aacctgtgac
ccctgcgcct ccacccagcg tgcgtgcacg tcctcc
SEQ ID NO: 4, RAB11B, Homo sapiens
MGTRDDEYDY LFKVVLIGDS GVGKSNLLSR FTRNEFNLES KSTIGVEFAT RSIQVDGKTI
KAQIWDTAGQ ERYRAITSAY YRGAVGALLV YDIAKHLTYE NVERWLKELR DHADSNIVIM
LVGNKSDLRH LRAVPTDEAR AFAEKNNLSF IETSALDSTN VEEAFKNILT EIYRIVSQKQ
IADRAAHDES PGNNVVDISV PPTTDGQKPN KLQCCQNL
SEQ ID NO: 5, Rab11a, Mus musculus
ggctcgtcac cgggtccggc agctgaagct cctcgctcgc tcgggttacc cctgcagcga
cgccccctgg tcccgccgcc gttgccaccg ccgctcccgc ccctcagctc ctcggccgcg
ccatgggcac ccgcgacgac gagtacgact acctctttaa agttgtcctt attggagatt
ctggtgttgg aaagagtaac ctcctgtctc gatttactcg aaatgagttt aatctggaaa
gcaagagtac cattggagta gagtttgcaa caagaagcat ccaggttgat gggaaaacaa
taaaggcaca gatatgggac acagcagggc aggagcggta cagggctata acgtctgcat
actatcgtgg agcagtaggt gccttattgg tttatgacat tgctaagcat ctcacatatg
aaaatgtaga gcgatggctg aaagaactga gagatcatgc tgatagtaac attgttatca
tgcttgtggg caataagagt gatttacgtc atctcagggc agttcctaca gatgaagcaa
gagcttttgc agagaagaat ggtttgtcat tcattgagac atctgctcta gattctacaa
atgttgaagc tgcttttcag acaattctaa cagagatata ccgcattgtt tctcagaagc
aaatgtcaga cagacgtgaa aatgacatgt ctccaagcaa caatgtggtt cctattcatg
ttccgcccac cactgagaac aagccaaagg tgcagtgctg tcagaacatc taaggcgtct
cttcccctag aaggctgtgt atagtccatt tcccaggtct gagatttaaa tatatttgta
attcttgtgg tcaaaaaaaa aaaaaaaaaa aaaaaaaaaa aaaaaaaaaa aaaaaaaaaa
aaa
SEQ ID NO: 6, Rab11a, Mus musculus
MGTRDDEYDY LFKVVLIGDS GVGKSNLLSR FTRNEFNLES KSTIGVEFAT RSIQVDGKTI
KAQIWDTAGQ ERYRAITSAY YRGAVGALLV YDIAKHLTYE NVERWLKELR DHADSNIVIM
LVGNKSDLRH LRAVPTDEAR AFAEKNGLSF IETSALDSTN VEAAFQTILT EIYRIVSQKQ
MSDRRENDMS PSNNVVPIHV PPTTENKPKV QCCQNI
SEQ ID NO: 7, Rab11b, Mus musculus
atggggaccc gggacgacga gtacgattac ctattcaaag tggtgcttat tggggactca
ggtgtaggta agagcaacct gctgtcacgc ttcaccagaa acgaattcaa cctagagagc
aagagtacca tcggagtgga gttcgccact cgcagcattc aggtggacgg caagaccatc
aaggctcaga tctgggacac tgctggccag gagcgctacc gtgccattac ctctgcgtac
taccgtggtg cagtgggtgc actgctggta tatgacattg ccaagcactt gacatatgag
aacgtggagc gctggctgaa ggagctgcgg gatcatgcag atagcaacat tgtcatcatg
ctggtgggca acaagagtga cctgcgccac cttcgggctg tgcccactga tgaggcccgt
gcctttgcag aaaagaacaa cttgtccttc attgagacct cagccttgga ttccaccaat
gtagaggaag cattcaagaa catcctcaca gaaatctacc gtattgtgtc acagaagcaa
atcgctgacc gtgcagccca cgatgagtcc cctggcaaca acgtggtgga catcagtgtg
ccacccacca ccgatggaca gagacccaac aagctgcagt gctgccagag cctgtga
SEQ ID NO: 8, Rabllb, Mus musculus
MGTRDDEYDY LFKVVLIGDS GVGKSNLLSR FTRNEFNLES KSTIGVEFAT RSIQVDGKTI
KAQIWDTAGQ ERYRAITSAY YRGAVGALLV YDIAKHLTYE NVERWLKELR DHADSNIVIM
LVGNKSDLRH LRAVPTDEAR AFAEKNNLSF IETSALDSTN VEEAFKNILT EIYRIVSQKQ
IADRAAHDES PGNNVVDISV PPTTDGQRPN KLQCCQSL
SEQ ID NO: 9, Yap1, Homo sapiens
atggatcccg ggcagcagcc gccgcctcaa ccggcccccc agggccaagg gcagccgcct
tcgcagcccc cgcaggggca gggcccgccg tccggacccg ggcaaccggc acccgcggcg
acccaggcgg cgccgcaggc accccccgcc gggcatcaga tcgtgcacgt ccgcggggac
tcggagaccg acctggaggc gctcttcaac gccgtcatga accccaagac ggccaacgtg
ccccagaccg tgcccatgag gctccggaag ctgcccgact ccttcttcaa gccgccggag
cccaaatccc actcccgaca ggccagtact gatgcaggca ctgcaggagc cctgactcca
cagcatgttc gagctcattc ctctccagct tctctgcagt tgggagctgt ttctcctggg
acactgaccc ccactggagt agtctctggc ccagcagcta cacccacagc tcagcatctt
cgacagtctt cttttgagat acctgatgat gtacctctgc cagcaggttg ggagatggca
aagacatctt ctggtcagag atacttctta aatcacatcg atcagacaac aacatggcag
gaccccagga aggccatgct gtcccagatg aacgtcacag cccccaccag tccaccagtg
cagcagaata tgatgaactc ggcttcagcc atgaaccaga gaatcagtca gagtgctcca
gtgaaacagc caccacccct ggctccccag agcccacagg gaggcgtcat gggtggcagc
aactccaacc agcagcaaca gatgcgactg cagcaactgc agatggagaa ggagaggctg
cggctgaaac agcaagaact gcttcggcag gcaatgcgga atatcaatcc cagcacagca
aattctccaa aatgtcagga gttagccctg cgtagccagt taccaacact ggagcaggat
ggtgggactc aaaatccagt gtcttctccc gggatgtctc aggaattgag aacaatgacg
accaatagct cagatccttt ccttaacagt ggcacctatc actctcgaga tgagagtaca
gacagtggac taagcatgag cagctacagt gtccctcgaa ccccagatga cttcctgaac
agtgtggatg agatggatac aggtgatact atcaaccaaa gcaccctgcc ctcacagcag
aaccgtttcc cagactacct tgaagccatt cctgggacaa atgtggacct tggaacactg
gaaggagatg gaatgaacat agaaggagag gagctgatgc caagtctgca ggaagctttg
agttctgaca tccttaatga catggagtct gttttggctg ccaccaagct agataaagaa
agctttctta catggttata g
SEQ ID NO: 10, Yap1, Homo sapiens
MDPGQQPPPQ PAPQGQGQPP SQPPQGQGPP SGPGQPAPAA TQAAPQAPPA GHQIVHVRGD
SETDLEALFN AVMNPKTANV PQTVPMRLRK LPDSFFKPPE PKSHSRQAST DAGTAGALTP
QHVRAHSSPA SLQLGAVSPG TLTPTGVVSG PAATPTAQHL RQSSFEIPDD VPLPAGWEMA
KTSSGQRYFL NHIDQTTTWQ DPRKAMLSQM NVTAPTSPPV QQNMMNSASA MNQRISQSAP
VKQPPPLAPQ SPQGGVMGGS NSNQQQQMRL QQLQMEKERL RLKQQELLRQ AMRNINPSTA
NSPKCQELAL RSQLPTLEQD GGTQNPVSSP GMSQELRTMT TNSSDPFLNS GTYHSRDEST
DSGLSMSSYS VPRTPDDFLN SVDEMDTGDT INQSTLPSQQ NRFPDYLEAI PGTNVDLGTL
EGDGMNIEGE ELMPSLQEAL SSDILNDMES VLAATKLDKE SFLTWL
SEQ ID NO: 11, Taz, Homo sapiens
cgcgcgctca ggctcagctt cgctgcccgc ccaggtagtg cccgctggag ctcgcgcgct
catccggcac cactccaggg ctccaggctc ctcgggcttc cggagtcgag acgtggtgga
gttggctcgg gctgaacttc tttcgggggg ctgcctgtcc ttctttttgc agaagatgaa
tccggcctcg gcgccccctc cgctcccgcc gcctgggcag caagtgatcc acgtcacgca
ggacctagac acagacctcg aagccctctt caactctgtc atgaatccga agcctagctc
gtggcggaag aagatcctgc cggagtcttt ctttaaggag cctgattcgg gctcgcactc
gcgccagtcc agcaccgact cgtcgggcgg ccacccgggg cctcgactgg ctgggggtgc
ccagcatgtc cgctcgcact cgtcgcccgc gtccctgcag ctgggcaccg gcgcgggtgc
tgcgggtagc cccgcgcagc agcacgcgca cctccgccag cagtcctacg acgtgaccga
cgagctgcca ctgcccccgg gctgggagat gaccttcacg gccactggcc agaggtactt
cctcaatcac atagaaaaaa tcaccacatg gcaagaccct aggaaggcga tgaatcagcc
tctgaatcat atgaacctcc accctgccgt cagttccaca ccagtgcctc agaggtccat
ggcagtatcc cagccaaatc tcgtgatgaa tcaccaacac cagcagcaga tggcccccag
taccctgagc cagcagaacc accccactca gaacccaccc gcagggctca tgagtatgcc
caatgcgctg accactcagc agcagcagca gcagaaactg cggcttcaga gaatccagat
ggagagagaa aggattcgaa tgcgccaaga ggagctcatg aggcaggaag ctgccctctg
tcgacagctc cccatggaag ctgagactct tgccccagtt caggctgctg tcaacccacc
cacgatgacc ccagacatga gatccatcac taataatagc tcagatcctt tcctcaatgg
agggccatat cattcgaggg agcagagcac tgacagtggc ctggggttag ggtgctacag
tgtccccaca actccggagg acttcctcag caatgtggat gagatggata caggagaaaa
cgcaggacaa acacccatga acatcaatcc ccaacagacc cgtttccctg atttccttga
ctgtcttcca ggaacaaacg ttgacttagg aactttggaa tctgaagacc tgatccccct
cttcaatgat gtagagtctg ctctgaacaa aagtgagccc tttctaacct ggctgtaatc
actaccattg taacttggat gtagccatga ccttacattt cctgggcctc ttggaaaaag
tgatggagca gagcaagtct gcaggtgcac cacttcccgc ctccatgact cgtgctccct
cctttttatg ttgccagttt aatcattgcc tggttttgat tgagagtaac ttaagttaaa
cataaataaa tattctattt tcattttcaa aaaaaaaaaa aaaaaaaaaa aaaaaaaaaa
aaaaaaaaaa aaaaaaaaaa aaaaaaaaaa aaaaaaaaaa aaaaa
SEQ ID NO: 12, Taz, Homo sapiens
MNPASAPPPL PPPGQQVIHV TQDLDTDLEA LFNSVMNPKP SSWRKKILPE SFFKEPDSGS
HSRQSSTDSS GGHPGPRLAG GAQHVRSHSS PASLQLGTGA GAAGSPAQQH AHLRQQSYDV
TDELPLPPGW EMTFTATGQR YFLNHIEKIT TWQDPRKAMN QPLNHMNLHP AVSSTPVPQR
SMAVSQPNLV MNHQHQQQMA PSTLSQQNHP TQNPPAGLMS MPNALTTQQQ QQQKLRLQRI
QMERERIRMR QEELMRQEAA LCRQLPMEAE TLAPVQAAVN PPTMTPDMRS ITNNSSDPFL
NGGPYHSREQ STDSGLGLGC YSVPTTPEDF LSNVDEMDTG ENAGQTPMNI NPQQTRFPDF
LDCLPGTNVD LGTLESEDLI PLFNDVESAL NKSEPFLTWL
SEQ ID NO: 13, Yap1, Mus musculus
cggacgcgtg gggccaaagt ttctgtctca gttgggacgc cgccgcggcc gggggcaaag
aaagggagga aggaaggagc tcgcggaggg gaggggagga gaggggaggc ggcctcgggc
aaggagtgca gggcgatgcg ggcgcgcgtc gcagcccccc gaacctgagc gcagtgcccc
gagcgtcgaa cgaggccgca gccatggagc ccgcgcaaca gccgccgccc cagccggccc
cgcaaggccc cgcgccgccg tccgtgtctc cggccgggac ccccgcggcc ccgcccgcac
ccccggccgg ccaccaggtc gtgcacgtcc gcggggactc ggagaccgac ttggaggcgc
tcttcaatgc cgtcatgaac cccaagacgg ccaacgtgcc tcagaccgtg cccatgcggc
ttcgcaagct gcccgactcc ttcttcaagc cgcctgagcc caagtcccac tcgcgacagg
ccagtactga tgcaggtact gcgggagctc tgactccaca gcatgttcga gctcactcct
ctccagcctc cctgcagctg ggtgccgttt ctcctgggac actcacagcc agtggcgttg
tctctggccc tgccgctgcc cctgcagctc agcatctccg gcagtcctcc tttgagatcc
ctgatgatgt accactgcca gcaggctggg agatggccaa gacatcttct ggtcaaagat
acttcttaaa tcacaacgat cagacaacaa catggcagga cccccggaag gccatgcttt
cgcaactgaa cgttcctgcg cctgccagcc cagcggtgcc ccagacgctg atgaattctg
cctcaggacc tcttcctgat ggatgggagc aagccatgac tcaggatgga gaagtttact
acataaacca taagaacaag accacatcct ggctggaccc aaggctggac cctcgttttg
ccatgaacca gaggatcact cagagtgctc cagtgaagca gcccccaccc ttggctcccc
agagcccaca gggaggcgtc ctgggtggag gcagttccaa ccagcagcag caaatacagc
tgcagcagtt acagatggag aaggagagac tgcggttgaa acaacaggaa ttatttcggc
aggcaatacg gaatatcaat cccagcacag caaatgctcc aaaatgtcag gaattagctc
tgcgcagcca gttgcctaca ctggagcagg atggagggac tccgaatgca gtgtcttctc
ctgggatgtc tcaggaattg agaacaatga caaccaatag ttccgatccc tttcttaaca
gtggcaccta tcactctcga gatgagagca cagacagcgg cctcagcatg agcagctaca
gcatccctcg gaccccagac gacttcctca acagtgtgga tgaaatggat acaggagaca
ccatcagcca aagcaccctg ccgtcacagc agagccgctt ccccgactac ctggaagccc
tccctgggac aaatgtggac cttggcacac tggaaggaga tgcaatgaac atagaagggg
aggagctgat gcccagtctg caggaagcgc tgagttccga aatcttggac gtggagtctg
tgttggctgc caccaagcta gataaagaaa gctttctcac gtggttatag agctgcaggg
agccactctg agtctgtgag ggatccacag agcctaagat gtgcacgcct gaaattcaga
taagtcagtg ggggttctct ggctaacaca gaaaacagat gaaccagtgt ccatcgttgt
tccgcttttc tctgcccgtc gctgctctta cgttggttgc tgacctcttc acggccggct
ctaaagaacc cgaaccgcag acagattcct ttgttaactc tgctatgata actacgttct
ctgggattgc tgggggatgg cctgctggat aatggatgtt ctgccttttg tccggtggtc
ctttcaccat cactttaact gaacacacag actgggaact gaatgctcta gaacattgtt
caagaggtgg tttcttcagc tgccttgggt ccaacaagcc agaggcattg cgtctgatct
cgtggaggac ggaggggacc cacgctgaag actggtgaac tttccattct tctgttagcg
atgccgttag gccatagtga cctgggtctt atcttagacg cttatgaggc atgagacagc
ttccatagaa atatattaat tattaccaca tactctagat taggtttgaa tgaatatttt
ctgtgggtgt tttggttggt ttttctctgc cccccccccc ttttttgtgg ttggtcttgg
tggaacgtag gcaaattaat gaattcgttt atagctgtag cttggggtgg gcaataccat
tcttttggtg ggaaatctgt atttcttggt tttttaacat cctatttaaa tcttaaatct
tggttatctc ctctctacat atatacacac tcttattatg tctatggtag tgtgatagca
gaatatatct ttataaacaa aaaaaaaaaa aaaaaaaaaa aaaaaaaaa
SEQ ID NO: 14, Yap1, Mus musculus
MEPAQQPPPQ PAPQGPAPPS VSPAGTPAAP PAPPAGHQVV HVRGDSETDL EALFNAVMNP
KTANVPQTVP MRLRKLPDSF FKPPEPKSHS RQASTDAGTA GALTPQHVRA HSSPASLQLG
AVSPGTLTAS GVVSGPAAAP AAQHLRQSSF EIPDDVPLPA GWEMAKTSSG QRYFLNHNDQ
TTTWQDPRKA MLSQLNVPAP ASPAVPQTLM NSASGPLPDG WEQAMTQDGE VYYINHKNKT
TSWLDPRLDP RFAMNQRITQ SAPVKQPPPL APQSPQGGVL GGGSSNQQQQ IQLQQLQMEK
ERLRLKQQEL FRQAIRNINP STANAPKCQE LALRSQLPTL EQDGGTPNAV SSPGMSQELR
TMTTNSSDPF LNSGTYHSRD ESTDSGLSMS SYSIPRTPDD FLNSVDEMDT GDTISQSTLP
SQQSRFPDYL EALPGTNVDL GTLEGDAMNI EGEELMPSLQ EALSSEILDV ESVLAATKLD
KESFLTWL
SEQ ID NO: 15, Taz, Mus musculus
gtccgggagc cgcggcggct gcgctcgtct acgtcttctc tgtcgcctcc tcgcgcagtg
ggagcgcccg aggccggttc cggggatgta agaggataag ccttcggctg ctgggaatcc
gctcgggatc tgcccgggac cgggttccag ctcgtcagtt cgggaggcgc ccaggcttgg
cttccccgag tccccagaaa gatgaatccg tcctcggtgc cccatccgct cccgccgcca
gggcagcaag tcatccacgt cacgcaggac ctggacaccg acctcgaagc cctcttcaac
tctgtcatga accccaagcc cagctcatgg cggaaaaaga tcctcccgga gtccttcttt
aaggagcccg attccggctc gcactcgcgc caatccagca cagactcatc aggcggccac
ccggggcctc gactagctgg cggcgcgcag cacgtccgct cgcactcgtc gcccgcatcc
ctgcagctgg gcaccggtgc gggagccgct ggaggccctg cacagcagca tgcacatctc
cgccagcagt cctatgacgt gaccgacgag ctgccgttgc cccccgggtg ggagatgacc
ttcacggcca ctggccagag atacttcctt aatcacatag agaaaatcac cacatggcaa
gaccccagga aggtgatgaa tcagcctctg aatcatgtga acctccaccc gtccatcact
tccacctcgg tgccacagag gtccatggca gtgtcccagc cgaatctcgc aatgaatcac
caacaccagc aagtcgtggc cactagcctg agtccacaga accacccgac tcagaaccaa
cccacagggc tcatgagtgt gcccaatgca ctgaccactc agcagcagca gcagcagaaa
ctgcggcttc agaggatcca gatggagaga gagaggatta ggatgcgtca agaggagctc
atgaggcagg aagctgccct ctgccgacag ctccccatgg aaaccgagac catggcccct
gtcaacacgc ctgccatgag cacagatatg agatctgtca ccaacagtag ctcagatcct
ttcctcaatg gagggcccta tcattcacgg gagcagagca cagacagtgg cctggggtta
gggtgctaca gtgtccccac aactccagaa gacttcctca gcaacatgga cgagatggat
acaggtgaaa attccggtca gacacccatg accgtcaatc cccagcagac ccgcttccct
gatttcctgg actgccttcc aggaacaaat gttgacctcg ggactttgga gtctgaagat
ctgatccctc tcttcaatga tgtagagtct gctctgaaca aaagcgagcc ctttctaacc
tggctgtaat cactactgtt gtaacgtgat gcagctgtga gctgacgcgc gtcttgggcc
ttgcggacca agtgatgagg cagagcgggc ctgcagctgc accacgttct gcctttgtac
tcacactcct tgtccgtgtg gccacttaat cattgcctgg tgttgattcg caggaacttg
cgttacacag aaataaatac tctcttttca ttttcaaaaa aaaaaaaaaa
SEQ ID NO: 16, Taz, Mus musculus
MNPSSVPHPL PPPGQQVIHV TQDLDTDLEA LFNSVMNPKP SSWRKKILPE SFFKEPDSGS
HSRQSSTDSS GGHPGPRLAG GAQHVRSHSS PASLQLGTGA GAAGGPAQQH AHLRQQSYDV
TDELPLPPGW EMTFTATGQR YFLNHIEKIT TWQDPRKVMN QPLNHVNLHP SITSTSVPQR
SMAVSQPNLA MNHQHQQVVA TSLSPQNHPT QNQPTGLMSV PNALTTQQQQ QQKLRLQRIQ
MERERIRMRQ EELMRQEAAL CRQLPMETET MAPVNTPAMS TDMRSVTNSS SDPFLNGGPY
HSREQSTDSG LGLGCYSVPT TPEDFLSNMD EMDTGENSGQ TPMTVNPQQT RFPDFLDCLP
GTNVDLGTLE SEDLIPLFND VESALNKSEP FLTWL

The indefinite articles “a” and “an,” as used herein in the specification and in the claims, unless clearly indicated to the contrary, should be understood to mean “at least one.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

It should also be understood that, unless clearly indicated to the contrary, in any methods claimed herein that include more than one step or act, the order of the steps or acts of the method is not necessarily limited to the order in which the steps or acts of the method are recited.

All references (e.g., published journal articles, books, etc.), patents and patent applications disclosed herein are incorporated by reference with respect to the subject matter for which each is cited, which, in some cases, may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of increasing mammalian cell expression of a protein of interest, comprising culturing mammalian cells that overexpress a protein of interest and are modified to overexpress a gene encoding Rab11 protein.

2. The method of claim 1, wherein the cells are cultured in cell culture media under conditions that permit production and secretion of the protein of interest into the media.

3. The method of claim 2, further comprising isolating and/or purifying the protein of interest from the media.

4. The method of any one of claims 1-3, wherein the mammalian cells comprise a recombinant nucleic acid encoding the Rab11 protein and/or a recombinant nucleic acid encoding the protein of interest.

5. The method of any one of claims 1-4, wherein the Rab11 protein is stably expressed in the mammalian cells.

6. The method of any one of claims 1-5, wherein the protein of interest is stably expressed in the mammalian cells.

7. The method of any one of claims 1-6, wherein the mammalian cells are Chinese hamster ovary (CHO) cells.

8. The method of any one of claims 1-7, wherein the Rab11 protein is a Rab11a isoform or a Rab11b isoform.

9. The method of claim 8, wherein the Rab11 protein is a Rab11b isoform.

10. The method of any one of claims 1-9, wherein the protein of interest is a therapeutic protein.

11. The method of claim 10, wherein the therapeutic protein is an antibody.

12. The method of claim 11, wherein the antibody is a monoclonal antibody.

13. The method of any one of claim 1-12, wherein the cell specific productivity of the mammalian cells is at least 50% greater than the cell specific productivity of mammalian cells that are not modified to comprise a nucleic acid encoding a Rab11 protein.

14. A mammalian cell that overexpresses a protein of interest and is modified to overexpress a gene encoding Rab11 protein.

15. The mammalian cell of claim 14, wherein the mammalian cell comprises a recombinant nucleic acid encoding the Rab11 protein and/or a recombinant nucleic acid encoding the protein of interest.

16. The mammalian cell of claim 14 or 15, wherein the Rab11 protein is stably expressed in the mammalian cell.

17. The mammalian cell of any one of claims 14-16, wherein the protein of interest is stably expressed in the mammalian cell.

18. The mammalian cell of any one of claims 14-17, wherein the mammalian cell is a Chinese hamster ovary (CHO) cell.

19. The mammalian cell of any one of claims 14-18, wherein the Rab11 protein is a Rab11a isoform or a Rab11b isoform.

20. The mammalian cell of claim 19, wherein the Rab11 protein is a Rab11b isoform.

21. The mammalian cell of any one of claims 14-20, wherein the protein of interest is a therapeutic protein.

22. The mammalian cell of claim 21, wherein the therapeutic protein is an antibody.

23. The mammalian cell of claim 22, wherein the antibody is a monoclonal antibody.

24. A method of producing modified mammalian cells, comprising:
modifying mammalian cells to express a Rab11 protein; and
introducing into the mammalian cells a recombinant nucleic acid encoding a protein of interest, thereby producing engineered mammalian cells that express Rab11 protein and comprise the recombinant nucleic acid encoding a protein of interest.

25. The method of claim 24, further comprising culturing the modified mammalian cells in media under conditions that permit production and secretion of the protein of interest into the media.

26. The method of claim 25, further comprising isolating and/or purifying the protein of interest from the media.

27. The method of any one of claims 24-26, wherein the step of modifying mammalian cells comprises introducing into the mammalian cells a recombinant nucleic acid encoding a Rab11 protein.

28. The method of any one of claims 24-27, wherein the Rab11 protein is stably expressed in the mammalian cells.

29. The method of any one of claims 24-28, wherein the protein of interest is stably expressed in the mammalian cells.

30. The method of any one of claims 24-29, wherein the mammalian cells are Chinese hamster ovary (CHO) cells.

31. The method of any one of claims 24-30, wherein the Rab11 protein is a Rab11a isoform or a Rab11b isoform.

32. The method of claim 31, wherein the Rab11 protein is a Rab11b isoform.

33. The method of any one of claims 24-32, wherein the protein of interest is a therapeutic protein.

34. The method of claim 33, wherein the therapeutic protein is an antibody.

35. The method of claim 34, wherein the antibody is a monoclonal antibody.

36. A method of increasing expression of a protein, comprising culturing mammalian cells that overexpress a protein of interest and are modified to overexpress a gene encoding Yap1 protein.

37. The method of claim 36, wherein the cells are cultured in cell culture media under conditions that permit production and secretion of the protein of interest into the media.

38. The method of claim 37, further comprising isolating and/or purifying the protein of interest from the media.

39. The method of any one of claims 36-38, wherein the mammalian cells comprise a recombinant nucleic acid encoding the Yap1 protein and/or a recombinant nucleic acid encoding the protein of interest.

40. The method of any one of claims 36-39, wherein the Yap1 protein is stably expressed in the mammalian cells.

41. The method of any one of claims 36-40, wherein the protein of interest is stably expressed in the mammalian cells.

42. The method of any one of claims 36-41, wherein the mammalian cells are Chinese hamster ovary (CHO) cells.

43. The method of any one of claims 36-42, wherein the Yap1 protein is a recombinant Yap1 protein.

44. The method of any one of claims 36-43, wherein the protein of interest is a recombinant protein of interest.

45. The method of any one of claims 36-44, wherein the protein of interest is a therapeutic protein.

46. The method of claim 45, wherein the therapeutic protein is an antibody.

47. The method of claim 46, wherein the antibody is a monoclonal antibody.

48. The method of any one of claim 36-47, wherein the cell specific productivity of the mammalian cells is at least 15% greater than the cell specific productivity of mammalian cells that are not modified to comprise a nucleic acid encoding a Yap1 protein.

49. A mammalian cell that overexpresses a protein of interest and a gene encoding Yap1 protein.

50. The mammalian cell of claim 49, wherein the mammalian cell comprises a recombinant nucleic acid encoding the Yap1 protein.

51. The mammalian cell of claim 49 or 50, wherein the Yap1 protein is stably expressed in the mammalian cell.

52. The mammalian cell of claim 49 or 51, wherein the protein of interest is stably expressed in the mammalian cell.

53. The mammalian cell of any one of claims 49-52, wherein the mammalian cell is a Chinese hamster ovary (CHO) cell.

54. The mammalian cell of any one of claims 49-53, wherein the Yap1 protein is a recombinant Yap1 protein.

55. The mammalian cell of any one of claims 49-54, wherein the protein of interest is a recombinant protein of interest.

56. The mammalian cell of any one of claims 49-55, wherein the protein of interest is a therapeutic protein.

57. The mammalian cell of claim 56, wherein the therapeutic protein is an antibody.

58. The mammalian cell of claim 57, wherein the antibody is a monoclonal antibody.

59. A method of producing modified mammalian cells, comprising:
modifying mammalian cells to express a Yap1 protein; and
introducing into the mammalian cells a recombinant nucleic acid encoding a protein of interest, thereby producing engineered mammalian cells that express Yap1 and comprise the recombinant nucleic acid encoding a protein of interest.

60. The method of claim 59, further comprising culturing the modified mammalian cells in media under conditions that permit production and secretion of the protein of interest into the media.

61. The method of claim 60, further comprising isolating and/or purifying the protein of interest from the media.

62. The method of any one of claims 59-61, wherein the step of modifying mammalian cells comprises introducing into the mammalian cells a recombinant nucleic acid encoding a Yap1 protein.

63. The method of any one of claims 59-62, wherein the Yap1 protein is stably expressed in the mammalian cells.

64. The method of any one of claims 59-63, wherein the protein of interest is stably expressed in the mammalian cells.

65. The method of any one of claims 59-64, wherein the mammalian cells are Chinese hamster ovary (CHO) cells.

66. The method of any one of claims 59-65, wherein the protein of interest is a therapeutic protein.

67. The method of claim 66, wherein the therapeutic protein is an antibody.

68. The method of claim 67, wherein the antibody is a monoclonal antibody.
